(19) United States
(12) Patent Application Publication
(10) Pub. No.: US 2023/0347164 A1
(43) Pub. Date: Nov. 2, 2023

(54) HOME PHOTOTHERAPY DEVICES AND ASSOCIATED SYSTEMS AND METHODS

(71) Applicant: BeneSol, Inc., Bainbriedge Island, WA (US)

(72) Inventors: Gary Lauder, Atherton, CA (US); Robert Wise, Bainbridge Island, WA (US); James Wu, Bellevue, WA (US); Tyson Colby, Bainbridge Island, WA (US); Brian M. Spahnie, Brunswick, OH (US)

(21) Appl. No.: 18/021,922

(22) PCT Filed: Aug. 18, 2021
(86) PCT No.: PCT/US2021/046578

§ 371 Date: Feb. 17, 2023

Related U.S. Application Data
(60) Provisional application No. 63/066,949, filed on Aug. 18, 2020.

(51) Int. Cl. A61N 5/06

(52) U.S. Cl. CPC A61N 5/0613; A61N 2005/0661; A61N 2005/0627; A61N 2005/0652; A61N 2005/0665

(57) ABSTRACT

A home phototherapy system for producing vitamin D via skin exposure and associated methods are disclosed herein. In some embodiments, the system includes a UV-emitting device that includes a housing and a UV light assembly carried by the housing. The UV light assembly can include an array of UVB light emitters and an optical component that, together, emit phototherapeutic UV radiation from an active side of the housing to promote vitamin D production in skin (e.g., a human torso) exposed to the UVB light. The system also includes a dose controller operably coupled to the UV light assembly that can create a dosing protocol for the UV light assembly and specific to the user. The dose controller can be implemented on an application in a mobile device, within the UV-emitting device, and/or a cloud server.

### CROSS-REFERENCE TO RELATED APPLICATION

This application claims the benefit of U.S. Provisional Application 63/066,949, filed on Aug. 18, 2020, and entitled HOME PHOTOTHERAPY DEVICES AND ASSOCIATED SYSTEMS AND METHODS, which is hereby incorporated by reference in its entirety.

### TECHNICAL FIELD

The present technology relates generally to at home phototherapeutic devices, systems, and methods.

### BACKGROUND

Vitamin D refers to a group of fat-soluble secosteriods that the human body can synthesize through adequate exposure to sunlight. More specifically, vitamin D3 is made in the skin when 7-dehydrocholesterol reacts with ultraviolet (“UV”) B light. Vitamin D can also be absorbed from the various dietary sources, such as fatty fish (e.g., salmon and tuna), vitamin D fortified foods (e.g., dairy and juice products), and vitamin D supplements. Once absorbed, the vitamin D travels through the bloodstream to the liver where it is converted into the prohormone calcidiol. The calcidiol is, in turn, converted into calcitriol (the hormonally active form of vitamin D) by the kidneys or monocyte-macrophages in the immune system. When synthesized by the monocyte-macrophages, calcitriol acts locally as a cytokine to defend the body against microbial invaders. Kidney-synthesized calcitriol circulates through the body to regulate the concentration of calcium and phosphate in the bloodstream, and thereby promotes adequate mineralization, growth, and reconstruction of the bones. Therefore, an inadequate level of vitamin D, (typically characterized by a calcidiol concentration in the blood of less than 20-40 ng/mL) can cause various bone softening diseases, such as rickets in children and osteomalacia in adults. Vitamin D deficiency has also been linked to numerous other diseases and disorders, such as depression, heart disease, gout, autoimmune disorders, and a variety of different cancers.

Recently, vitamin D deficiency has become a prominent condition due, at least in part, to increasingly metropolitan populations and the resultant indoor lifestyles that inhibit adequate daily exposure to sunlight for vitamin D production. The growing emphasis on skin cancer awareness and sunscreen protection, which blocks UVB rays, may have also increased the spread of vitamin D deficiency. Additionally, various environmental factors, such as geographic latitude, seasons, and smog, further impede sufficient vitamin D production.

Physicians have recommended vitamin D supplements as a preventative measure to increase vitamin D levels. The American Institute of Medicine, for example, recommends a daily dietary vitamin D intake of 600 international units (IU) for those 1-70 years of age, and 800 IU for those 71 years of age and older. Other institutions have recommended both higher and lower daily vitamin D doses. The limitations on daily dosages also reflect an effort to prevent ingesting too much vitamin D, which can eventually become toxic. In contrast, the human physiology has adapted to significantly higher daily doses of vitamin D from sunlight (e.g., 4,000-20,000 IU/day or more). UVB radiation has been identified as a more desirable source of vitamin D because of the ease at which vitamin D is produced from exposure to sunlight and the body's natural ability to inhibit excessive vitamin D intake through the skin.

### BRIEF DESCRIPTION OF THE DRAWINGS

FIG. 1A illustrates a portable phototherapy system with a partial cutaway isometric view of a UV emission device configured in accordance with some embodiments of the present technology.

FIG. 1B is a network diagram of the portable phototherapy system of FIG. 1A in accordance with some embodiments of the present technology.

FIG. 2A is a cross sectional view of a component of a UV light assembly configured in accordance with some embodiments of the present technology.

FIG. 2B is an isometric front view of a section of the UV light assembly of FIG. 2A configured in accordance with some embodiments of the present technology.

FIG. 2C is an isometric back view of the section of the UV light assembly of FIG. 2B configured in accordance with some embodiments of the present technology.

FIG. 2D is top of the section of the UV light assembly of FIG. 2B configured in accordance with some embodiments of the present technology

FIG. 3 is a side view illustrating a dynamic dosing device emitting focused UV light on a user in accordance with some embodiments of the present technology.

FIG. 4 is an illustration of a Fitzpatrick skin tone selection GUI in accordance with embodiments of the present technology.

FIG. 5 is a flow diagram illustrating a process for calibrating a dose of UV emissions for a user in accordance with some embodiments of the present technology.

FIG. 6 illustrates dosing tables for skin types for increasing dosages and at varying stages of the calibration process of FIG. 5 in accordance with some embodiments of the present technology.

FIG. 7 is a flow diagram of an authentication process for a dynamic dosing system configured in accordance with some embodiments of the present technology.

FIG. 8A is a side view of a component in a UV light assembly configured in accordance with some embodiments of the present technology.

FIG. 8B is a top view of a section of the UV light assembly of FIG. 8A configured in accordance with some embodiments of the present technology.

FIG. 9A is a side view of a component in a UV light assembly configured in accordance with some embodiments of the present technology.

FIG. 9B is a top view of a section of the UV light assembly of FIG. 9A configured in accordance with some embodiments of the present technology.

FIG. 10A is a side view of a component in a UV light assembly configured in accordance with some embodiments of the present technology.

FIG. 10B is a top view of a section of the UV light assembly of FIG. 10A configured in accordance with some embodiments of the present technology.

FIG. 11A is a front view of a UV-emitting device for a portable phototherapy system in accordance with some embodiments of the present technology.

FIG. 11A is a rear view of the UV-emitting device of FIG. 11A in accordance with some embodiments of the present technology.

The drawings have not necessarily been drawn to scale. Similarly, some components and/or operations can be separated into different blocks or combined into a single block for the purpose of discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

A home phototherapy system (also referred to as a “portable phototherapy system”) for producing vitamin D via skin exposure to UVB radiation and associated systems and methods are disclosed herein. In some embodiments, the system includes a UV-emitting device that includes a housing and a UV light assembly within the housing. The UV light assembly can include an array of UV light emitters positioned to emit phototherapeutic UV radiation and, in some embodiments, an optical component (e.g., reflectors, lenses, and other suitable optics) component between the UV light emitters and an active side of the housing. The optical component can be sized and shaped to direct and/or focus phototherapeutic UV radiation toward the active side of the housing and outwardly to a phototherapy zone a distance away from the active side.

The system can also include a dose controller operably coupled to the UV light assembly. The dose controller can be configured to execute a dose-defining protocol to identify a skin type for the user, identify a minimal erythemal dose (MED), and/or define a dosing protocol for the UV light assembly based on the user's skin type and/or MED. The dosing protocol delivers a calibrated dose of UV radiation to promote vitamin D production via a user's skin while limiting a total UV exposure based on the user's UV tolerance skin. In some embodiments, the dose controller is configured to implement an authentication protocol that confirms the user's identity before each phototherapy session and, therefore, avoids exposing others to UV radiation not specific to their therapy protocol. For example, the authentication protocol can prevent a second user from receiving a UV dose specific to a first user, which may be above the second user's UV tolerance. In another example, the authentication protocol can prevent UV exposure to an unintended person (e.g., a child near the UV-emitting device).

In some embodiments, the dose controller can be implemented by a platform on a cloud server. For example, a user can access the dose controller through a personal electronic device (“PED,” such as a smartphone, tablet, laptop computer, personal computer, desktop computer, personal assistant, and the like). In such embodiments, the PED can communicate with the cloud server (e.g., via a network connection) to prompt the cloud server to execute the dose-defining protocol and/or the authentication protocol. The cloud server can then communicate a resulting dosing protocol to the PED, which can then relay the dosing protocol and/or a confirmation of authentication to a relevant UV-emitting device (e.g., using a network connection and/or a short-range wireless (e.g., Bluetooth®) connection). The UV-emitting device can receive the dosing protocol and/or the confirmation of authentication and deliver a dose of UV exposure in accordance with the dosing protocol. In some embodiments, the PED identifies the UV-emitting device when prompting the cloud server to execute the dosing protocol and/or the authentication protocol. In some such embodiments, the cloud server communicates the dosing protocol and/or the confirmation of authentication directly to the identified UV-emitting device.

In various embodiments, the dose controller can be implemented in various other locations. For example, in some embodiments, the PED includes an application that implements the dose controller locally on the PED. In such embodiments, the user can prompt their PED to execute the dose-defining protocol and/or the authentication protocol and communicate the results directly to the UV-emitting device. In some embodiments, the UV-emitting device itself includes electronic components to implement (or at least partially implement) the dose controller. In such embodiments, the user can access the UV-emitting device (e.g., via an onboard touchscreen or through the PED) to prompt the UV-emitting device to execute the dose-defining protocol and/or the authentication protocol.

Specific details of several embodiments of the present technology are described herein with reference to drawings. Although many of the embodiments are described with respect to devices, systems, and methods for phototherapy systems for stimulating vitamin D production via the skin, other embodiments in addition to those described herein are within the scope of the present technology. For example, at least some embodiments of the present technology may be useful for the treatment of various indications, such as skin diseases (e.g., psoriasis) and autoimmune diseases. Furthermore, at least some embodiments of the present technology may be used to provide preventative therapies. It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

Selected Components of the Phototherapy System

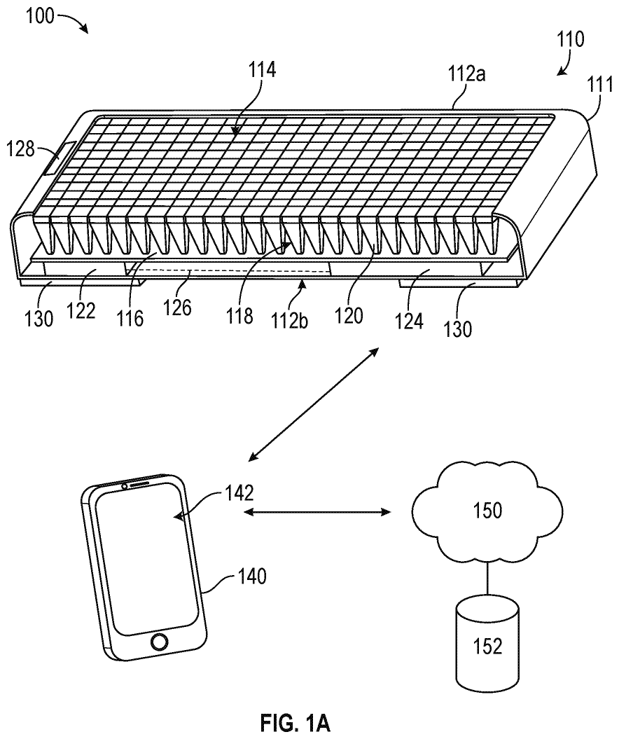

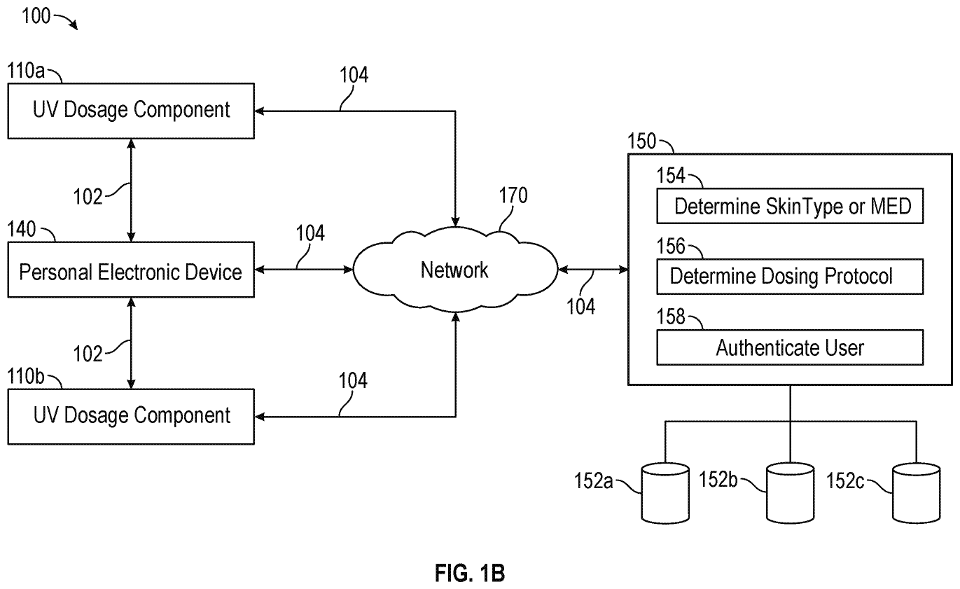

FIG. 1A illustrates a portable phototherapy system 100 (“system 100”) with a partial cutaway isometric view of a UV emission device configured in accordance with some embodiments of the present technology. The system 100 includes a UV-emitting device 110 housing 111 that includes a housing 111 with an active side 112a and a mounting side 112b (also referred to as “first side 112a” and “second side 112b,” respectively), a UV light assembly 114 within the housing 111 that includes an array of UV light emitters 116 and an optical component 118 (e.g., a reflector component, total internal reflection lens (TIR lens), other optical lens, and the like) positioned such that at least some of the emissions from the array of UV light emitters 116 are directed by the optical component 118 before exiting the system 100 via the active side 112a. As a result, and as discussed in more detail below, the optical component 118 can improve the uniformity of an average irradiance and/or distribution of the phototherapeutic UV radiation in a phototherapy zone. In the illustrated embodiment, the optical component 118 is an array of reflection components 120 individually corresponding to the array of UV light emitters 116 and positioned to at least partially collimate and/or direct light from the array of UV light emitters 116. In various other embodiments, the optical component 118 can be an array of TIR lenses and/or other suitable optical lenses that at least partially collimate and/or direct light from the array of UV light emitters 116. In this way, the UV light assembly 114 is configured to emit phototherapeutic UV radiation toward the active side 112a and outwardly towards a user of the system 100.

In some embodiments, the housing 111 can be a waterproof material and can be sealed on the active surface to protect the UV light assembly 114 within the housing 111. For example, a waterproof housing 111 can allow the system 100 to be used in the shower to minimize disruption to the user's ordinary routine. In some embodiments, the housing 111 can also be a shock-resistant material to provide mechanical protection to the UV light assembly 114. In some embodiments, the UV-emitting device 110 can be sized to be relatively portable. For example, in some embodiments, the UV-emitting device 110 can have dimensions of about 30 inches (″) in width, about 30″ in length, and about 4″ in thickness. In some embodiments, the UV-emitting device 110 can have dimensions ranging from about 5″ to about 50″ in width, about 5″ to about 50″ in length, and about 1″ to about 8″ in thickness. Further, in some embodiments, the width and length are not equal dimensions. For example, in some embodiments, the UV-emitting device 110 can have dimensions of about 20″ in width and about 25″ in length. As discussed in more detail below, the size of the housing 111 can also be selected to improve the uniformity of an average irradiance and/or distribution of the phototherapeutic UV radiation in a phototherapy zone.

In various embodiments, the array of UV light emitters 116 can emit phototherapeutic UV radiation having a peak wavelength between about 285 nanometers (nm) to about 315 nm, from about 293 nm to about 299 nm, or of about 297 nm. In some embodiments, the array of UV light emitters 116 can be an array of light emitting diodes (LEDs) configured to emit the phototherapeutic UV radiation. In some embodiments, the array of UV light emitters 116 can be a microplasma film containing an array of microcavities configured to emit the phototherapeutic UV radiation. In various embodiments, the array of UV light emitters 116 can be various other light emitting panels configured to emit UV radiation.

In the illustrated embodiment, the UV-emitting device 110 also includes a first electrical component 122 and a second electrical component 124 operably coupled to the UV light assembly 114 through one or more connection channels 126. In some embodiments, the first electrical component 122 can be connected to a data gathering component 128 (e.g., a camera configured to obtain images of the user's skin and/or the user's face, a touch screen to display information and receive inputs from a user, etc.).

In some embodiments, the first electrical component 122 can include a dose controller configured to execute a dose-defining protocol to define a dosing protocol for delivering a dose of UV radiation from the UV light assembly 114 to the user to promote vitamin D production via the user's skin while limiting the user's exposure to the UV radiation to a safe level. In some embodiments, for example, the dose controller can analyze images of the user's skin received from the data gathering component 128 in defining the dosing protocol. Additionally, or alternatively, the dose controller in the first electrical component 122 can be configured to define an authentication protocol that controls access to UV radiation from the system 100. For example, the authentication protocol can use biometric data obtained from the data gathering component 128 to confirm that the user is a registered user in the system 100 to reduce the likelihood of unintentional radiation exposure to unknowing parties (e.g., to reduce the chance a child will accidentally be exposed to the UV radiation, reduce the chance an adult is exposed to the UV radiation without knowing what light they are turning on, etc.). Each of these functions are discussed in more detail below. In some embodiments, the first electrical component 122 is configured to receive and execute the dosing protocol and/or to require a confirmation from the authentication protocol before executing the dosing protocol. For example, as discussed in more detail below, the dose-defining protocol and/or the authentication protocol can be executed by another component of the system 100. The first electrical component 122 can then receive instructions for executing the dosing protocol to deliver a dose of UV radiation to a user.

In some embodiments, the dose controller can also include a feedback mechanism to respond to images of the user's skin and adjust the dosing protocol. For example, in some embodiments, the dose controller (either via the first electrical component 122 or any other suitable component) can analyze an image of the user's skin and/or feedback inputs from the user before each dose to check for harmful effects from the radiation and reduce the dose if any is found.

In some embodiments, the second electrical component 124 can be a power source for the UV light assembly 114. In some embodiments, the second electrical component 124 can be an on-board battery. In some embodiments, the second electrical component 124 can be coupled to an exterior power source through a power cord.

In the illustrated embodiment, the UV-emitting device 110 also includes mounting elements 130, which are depicted schematically on the mounting side 112b of the housing 111. In some embodiments, the mounting elements 130 can be suction cups configured to hold the housing 111 against a wall or other surface. For example, the suction cups can allow the UV-emitting device 110 to be used in a bathroom (e.g., in the shower, on a mirror, and the like) during a user's morning routine (e.g., while they shower, shave, and/or get dressed). In various other embodiments, the mounting elements can be various other mechanical elements configured to hold the housing 111 in place for the user such as magnets, Command® strips, one or more hooks, one or more hanging bars, one or more brackets, and the like, that allow the user to mount the device in any other location for convenient use (e.g., in a closet or bedroom). In still other embodiments, the system 100 can include additional or alternative mounting elements on other surfaces of the housing 111. In some embodiments, the UV-emitting device 110 can include a stand (not shown) in place of, or in addition to, the mounting elements. In these embodiments, the stand can allow the housing 111 to be positioned to provide a dose to the user without attaching to another object or wall for support. In some embodiments, the mounting elements 130 include features that allow the height of the UV-emitting device 110 to be adjusted, thereby adjusting the elevation of the UV light assembly 114. That is, the height adjustment features allow the elevation of the radiation emitting components to be adjusted to be tailored to the user. For example, the height adjustment features allow a taller first user to raise the elevation of the UV-emitting device 110 and a shorter second user to lower the elevation of the UV-emitting device 110.

In some embodiments, the system 100 can include multiple UV-emitting devices 110, which can be positioned to deliver a dose of UV radiation from multiple angles and/or to multiple surfaces of the user's skin at one time (e.g., a user's front and back, a user's sides, etc.). Additionally, or alternatively, the multiple UV-emitting devices 110 can be in various convenient locations. For example, a first UV-emitting device can be positioned in a shower, while a second UV-emitting device can be positioned in a bedroom. Further, multiple UV-emitting devices 110 in the system 100 are in dispersed geographic locations (e.g., the user's gym, home, spa, in various hotels, in multiple homes or apartments, etc.). That is, the system 100 can connect any number of dispersed UV-emitting devices 110, allowing the user to receive a dose of UV radiation in any suitable location.

In the illustrated embodiment, the system 100 also includes a personal electronic device 140 (“PED”) in communication with the UV-emitting device 110 and a cloud server 150 in communication with the PED 140. The PED 140 includes an application 142 with a user interface allowing the user to interact with the UV-emitting device 110 and/or the cloud server 150. In some embodiments, the cloud server 150 includes one or more databases 152 (one shown in FIG. 1A) storing computer-executable instructions to implement the dose controller to execute the dose-defining protocol to: identify a skin type for the user; identify a minimal erythemal dose (MED); define a dosing protocol for the UV light assembly based on the user's skin type, MED, time since a previous dose, and/or a reaction to a previous dose; and/or implement the authentication protocol to confirm the user's identity before a dose of UV radiation is delivered. In some such embodiments, the PED 140 acts as an intermediary between the cloud server 150 and the UV-emitting device 110. For example, the user can prompt the cloud server 150 to define the dosing protocol through the application 142 on the PED 140. Before doing so, the cloud server 150 can execute the authentication protocol to confirm the identity of the user. Once confirmed, the cloud server 150 can define and send the dosing protocol to the PED 140. The user can then send the dosing protocol to the UV-emitting device 110 through the application 142 on the PED 140. Once the UV-emitting device 110 receives the dosing protocol, the UV-emitting device 110 delivers the relevant dose of UV radiation.

In some embodiments, the dose controller can operate at least partially on the PED 140 and/or the UV-emitting device 110 in addition to, or in alternative to, the cloud server 150. For example, the PED 140 can receive inputs related to the user's skin type and/or MED, use the inputs to identify the user's skin type and/or MED, then communicate the skin type and/or MED to the cloud server 150 for use in defining the dosing protocol. In another example, the UV-emitting device 110 can be configured to implement the authentication protocol after receiving the dosing protocol and before delivering the corresponding dose of UV radiation.

In some embodiments, the PED 140 can be a device associated with the user and communicably linked to the housing 111. For example, in various embodiments, the PED 140 can be a smartphone, tablet, laptop computer, personal computer, desktop computer, personal assistant, and/or any other suitable electronic device. In some embodiments, the PED 140 can be a detachable component of the housing 111, rather than a device specific to the user. For example, in some embodiments, the housing 111 can include a touch screen device (not shown) running the user application 142. The touch screen device can be permanently embedded in the housing 111, or can be removably attached to the housing 111.

FIG. 1B is a network diagram of the system 100 of FIG. 1A in accordance with some embodiments of the present technology. In the illustrated embodiment, the system 100 includes two UV-emitting devices 110 (referred to individually as a “first UV-emitting device 110a” and a “second UV-emitting device 110b”), the PED 140, and the cloud server 150. In the illustrated embodiment, the PED 140 can communicate with either of the UV-emitting devices 110 through one or more first communication channels 102 (e.g., based on short-range wireless communication connections such as Bluetooth®, Zigbee®, Z-Wave®, Wi-Fi HaLow®, and the like). Meanwhile, each of the UV-emitting devices 110, the PED 140, and the cloud server 150 can communicate with a network 170 (e.g., the internet) via second communication channels 104 (e.g., through a WiFi connection and/or a cellular connection). Accordingly, each of the UV-emitting devices 110, the PED 140, and the cloud server 150 can communicate with each other through the network 170.

Accordingly, in the illustrated embodiment, the cloud server 150 can communicate with the UV-emitting devices 110 without needing to relay through the PED 140. However, the cloud server 150 needs to know which of the first and second UV-emitting devices 110a, 110b to communicate with. Accordingly, in some embodiments, the PED 140 can identify which of the first and second UV-emitting devices 110a, 110b the user intends to use when prompting the cloud server 150 to execute the dose-generating protocol and/or the authentication protocol. In some embodiments, the identification can include providing the cloud server 150 with a device ID and/or IP address unique to the first and second UV-emitting devices 110a, 110b. In some embodiments, the first and second UV-emitting devices 110a, 110b communicate their device ID and/or IP address to the PED 140 through the first communication channels 102. In some embodiments, the first and second UV-emitting devices 110a, 110b include visible identifiers (e.g., signs, QR codes, and the like) that communicate their device ID and/or IP address. In some embodiments, the device ID and/or IP address for the first and second UV-emitting devices 110a, 110b can be saved in the PED 140 and/or the cloud server 150, allowing the user to select the relevant UV-emitting device from a list of the saved devices.

As further illustrated in FIG. 1B, the cloud server 150 can include multiple databases 152 (three illustrated, first-third databases 152a-152c) and one or more modules (three shown, referred to individually as a “first module 154,” a “second module 156,” and a “third module 158” and collectively as the “first-third modules 154-158”). The databases 152 can store information about the user, instructions for executing the first-third modules 154-158, the device ID and/or IP address for one or more UV-emitting devices 110, and/or any other suitable information.

The first and second modules 154, 156 are also sometimes referred to collectively as the dose-defining protocol. In the first module 154, the cloud server 150 can determine the user's skin type, MED, and/or an initial MED. As discussed in further detail below, the user's skin type can be determined based on a number of factors, such as the user's typical response to UV radiation (e.g., whether the user burns, freckles, tans, or has no reaction), their skin tone, etc. The user's skin type is commonly correlated with an appropriate MED for the user, which can then be used to define a dosing protocol for the user. In some embodiments, the cloud server 150 determines an initial MED for the user that reflects a confidence level in the skin type determination and/or the MED determination. For example, when the confidence level is low, the initial MED can be a fraction of the estimated MED to avoid causing an erythemal reaction. In some embodiments, the cloud server 150 determines an initial MED that is a screening dose. The screening dose can help detect outliers that have factors (e.g., skin tone) associated with an estimated MED, but have an actual MED that is lower than the estimated MED even when the confidence in the estimated MED is high.

In the second module 156, the cloud server 150 can determine the dosing protocol with an appropriate dose of UV radiation. Determining the dosing protocol can include determining a first dose based on the user's skin type and/or the initial MED, as well as determining any subsequent doses. As discussed in more detail below, determining subsequent doses can include receiving inputs from the user related to their response to the previous dose of UV radiation. For example, if the user experiences no erythema symptoms, the cloud server 150 can increase the amount of UV radiation delivered. Conversely, if the user experiences erythema symptoms, the cloud server 150 can decrease the amount of UV radiation delivered.

In the third module 158, the cloud server 150 can authenticate the user before sending any determined dosing protocol. To authenticate the user, the cloud server 150 can receive inputs such as user credentials (e.g., a username and password), and identifier associated with the PED 140 that confirms the identity of the user, biometric information, and/or any other information confirming the identity of the user. Authenticating the user can help ensure reduce the number of accidental exposures to the UV radiation emitted from the UV-emitting devices 110. For example, the authentication can help ensure that a first user dose not receive a dose associated with the dosing protocol of a second user with a higher MED and/or help ensure that a person (e.g., a child) does not unintentionally activate the UV-emitting devices 110. In some embodiments, the authentication helps ensure that an appropriate amount of time has passed since the user's last dose.

The first-third modules 154-158 are associated with the functions of the dose controller in the system 100. As discussed above, in some embodiments, the dose controller is at least partially implemented on the PED 140 and/or the UV-emitting devices 110. Purely by way of example, in some embodiments, the PED 140 can implement the third module 158 to authenticate the user before any of the UV-emitting devices 110 are activated.

In some embodiments, the cloud server 150 (and/or the PED 140 and the UV-emitting devices 110) include one or more additional, or alternative, modules. For example, the cloud server 150 can include a module that tracks the doses of UV radiation the user receives and/or sends reminders to the user when it is time for another dose. The reminders can include various notifications (e.g., push notifications, text messages, emails, and the like) that are delivered to the user through the PED 140 and/or any other suitable device. The tracking module can also keep a record of the UV doses for review by the user to track their progress, share with a medical care provider, and/or evaluate how their mood and/or health has fluctuated with the doses.

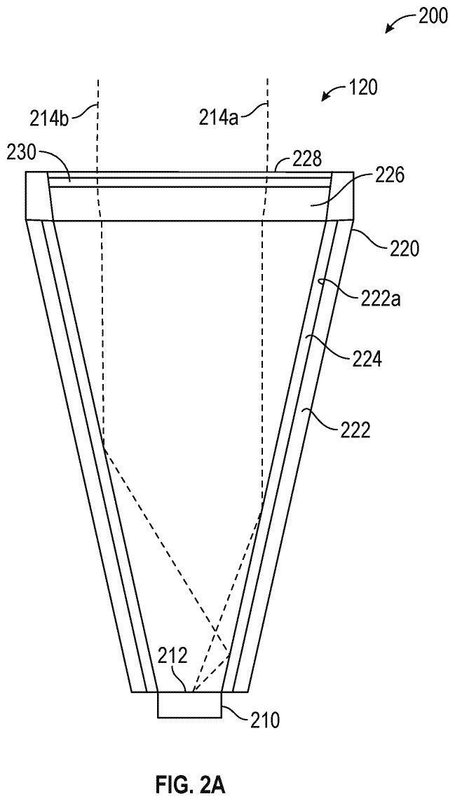

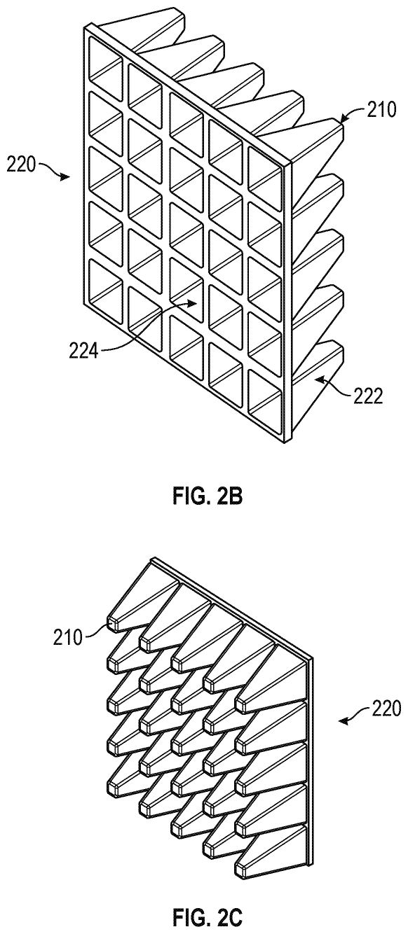

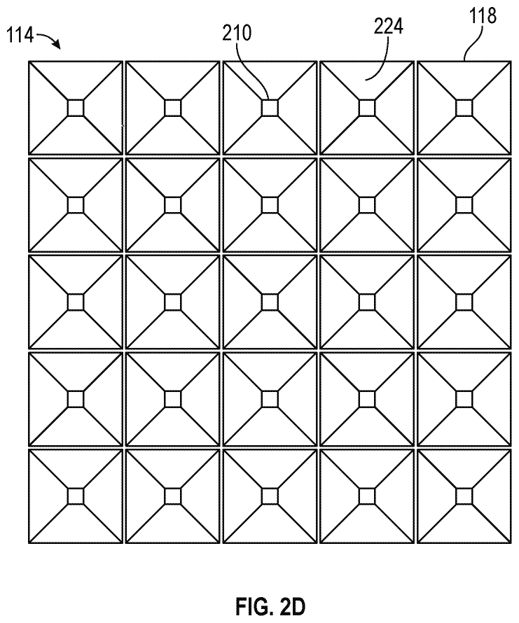

FIGS. 2A-2D illustrate further details on the UV light assembly 114, in accordance with some embodiments of the present technology. More specifically, FIGS. 2A-2D illustrate details on one embodiment of the optical component 118 attached to the array of UV light emitters 116 in the embodiment illustrated in FIG. 1A.

FIG. 2A is a cross sectional view of a component 200 of a UV light assembly 114 configured in accordance with some embodiments of the present technology. In the illustrated embodiment, the component 200 includes an LED 210 and an optical reflector 220 attached an active side 212 of the LED 210. The optical reflector 220 includes a body 222 having a horn or pyramid shape with an interior surface 222a that is covered by a reflective coating 224, an optional lens 226, a protective cover 228 (e.g., an acrylic layer of material), and an optional filter 230 between the optional lens 226 and the protective cover 228. In various embodiments, the body 222 can be made from a pliable, semi-rigid, and/or rigid UV-resistant materials (e.g., plastic resin, metal) that is molded, 3D printed, or can be made from independently constructed walls adhered together. In various embodiments, the reflective coating can be an aluminum coating and/or other suitable reflective coating.

As further illustrated by FIG. 2A, the body 222 has a geometry that is configured to reflect light emitted from the LED 210 into generally parallel travel paths 214. For example, a first travel path 214a travels outwardly from the active side 212 of the LED 210 and contacts the reflective coating 224. The angle of the body 222, and therefore the reflective coating 224 covering it, directs the first travel path into a direction generally orthogonal to the active side 212. Finally, in some embodiments, the travel path 214a passes through the optional lens 226 and the optional filter 230. In some embodiments, for example, the lens 226 can be a collimating lens where the first travel path 214a is further corrected into the generally orthogonal direction. In some embodiments, the optional filter 230 can be a high pass filter that blocks light emitted from the LED 210 with a wavelength below about 290 nm. In some embodiments, the optional filter 230 can be a low pass that blocks light emitted from the LED 210 with a wavelength above about 305 nm. In some embodiments, the optional filter 230 can be a band pass filter that blocks light emitted from the LED 210 with a wavelength outside of a range of from about 293 nm to about 303 nm. A second travel path 214b is also illustrated by FIG. 2A. As illustrated, the second travel path 214b initially travels outwards at a more acute angle than the first travel path 214a. Accordingly, the second travel path 214b quickly contacts the reflective coating 224 and is directed at least partly towards the generally orthogonal direction. However, the second travel path 214b contacts the reflective coating 224 a second time generally opposite the first contact point and is further deflected towards the generally orthogonal direction before reaching the lens 226 for a final correction. In this way, the optical reflector 220 is able to direct a substantial portion of the light emitted from the LED 210 into the generally orthogonal direction (and therefore into travel paths 214 that are generally parallel to each other).

FIGS. 2B-2D are isometric views of a section of the UV light assembly 114 of FIG. 2A configured in accordance with some embodiments of the present technology. In the illustrated embodiment, the section of the UV light assembly 114 includes a five-by-five (5×5) array of LEDs 210 connected to a five-by-five (5×5) array of components 200 (FIG. 2A). In some embodiments, the UV light assembly 114 can be manufactured in five-by-five sections that are then interconnected to form the complete UV light assembly 114 to improve manufacturing speeds and convenience. In various other embodiments, the UV light assembly 114 can be manufactured in various other sized arrays ranging from a single component to the entire assembly altogether. In some embodiments, the array of light emitters (e.g., an array of LEDs) can be manufactured separately from the reflector components (e.g., an array of optical lenses) that are connected after each element is complete.

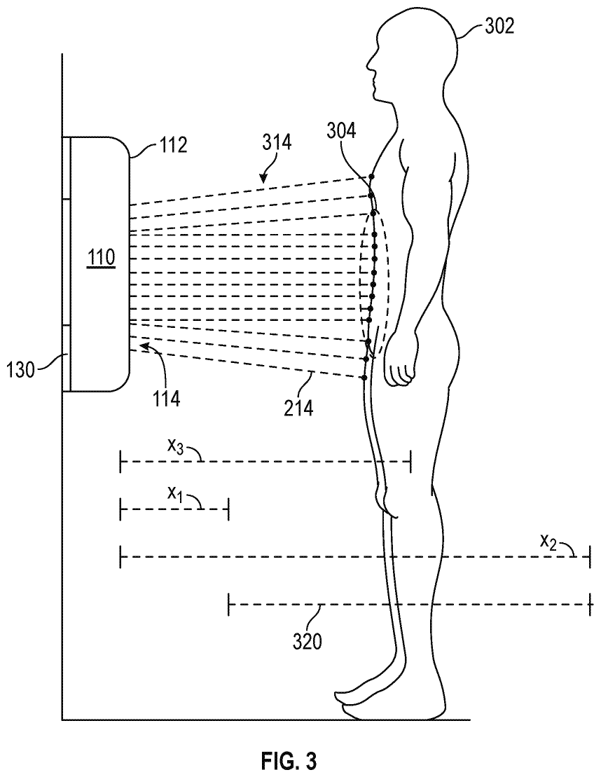

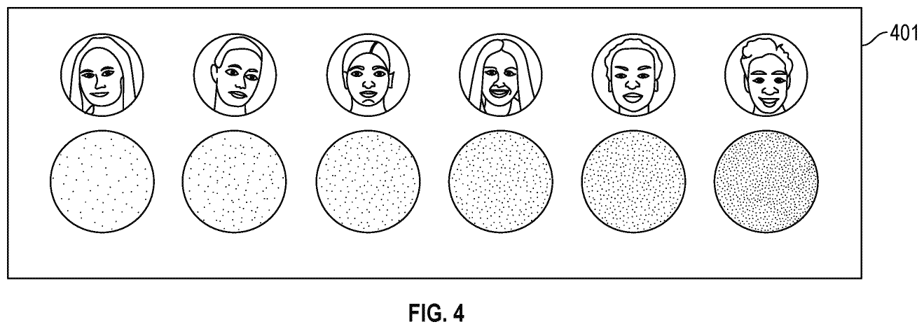

FIG. 3 is a side view illustrating a dynamic dosing device emitting focused UV light on a user 302 in accordance with some embodiments of the present technology. As illustrated, the system 100 delivers phototherapeutic UV radiation 314 to a treatment area 304 on the user 302 positioned within a phototherapy zone 320 a distance away from the active side 112a of the housing 111. In some embodiments, the treatment area 304 can be the front or backside of the user's torso. In some embodiments, the treatment area 304 can be a smaller or larger portion of the user 302. In some embodiments, the user 302 can vary the treatment area 304 for different doses. For example, the user 302 may expose their front torso for a first bi-weekly treatment and the backside of their torso for a second bi-weekly treatment.

As further illustrated in FIG. 3, the phototherapeutic UV radiation 314 is comprised of numerous beams having their own travel path 214 directed outwardly from the UV light assembly 114 towards the phototherapy zone 320. The travel paths 214 gradually disperse as they get farther from the active side 112a and, therefore, the average irradiance of the phototherapeutic UV radiation 314 gradually goes down farther from the active side 112a while the distribution of the phototherapeutic UV radiation 314 gradually becomes less uniform. As a result, the system 100 is most effective when the user 302 positions the treatment area 304 within the phototherapy zone 320. In the illustrated embodiment, the system has an optimized distance for the phototherapy zone 320 that ranges from a first distance X₁ to a second distance X₂ away from the active side 112a of the housing 111. In the phototherapy zone 320, the uniformity of the radiation is less than +1-10% from the average irradiance of the radiation at a third distance X₃ (e.g., a target distance) away from the active side 112a of the housing 111. In some embodiments, the phototherapy zone 320 ranges from about 3″ to about 21″ away from the active side 112a, with a target distance X₃ of about 12″ away from the active side 112a.

It will be appreciated that the range (e.g., from X₁ to X₂) of the phototherapy zone 320 is affected by the area of the phototherapeutic UV radiation 314 leaving the active side 112a. For example, a larger area leaving the active surface will maintain an average irradiance in the treatment area 304 at farther distances. Accordingly, the size of the housing 111 and the area of the UV light assembly 114 therein can be varied at least partly based on the desired distance of the phototherapy zone 320.

As disclosed above, in some embodiments, the system 100 can include multiple dynamic dosing devices emitting focused UV light on the user 302 (not shown). For example, in some embodiments, the system 100 can include a second housing positioned to emit phototherapeutic UV radiation towards a treatment area on the user's back simultaneously with the illustrated housing 111 emitting phototherapeutic UV radiation 314 onto the user's front. In addition, or alternatively, the multiple devices can be positioned to emit the phototherapeutic UV radiation towards the user's sides, and/or any other suitable treatment area of the user's skin.

Selected Phototherapy Methods for Vitamin D Production

Before receiving phototherapeutic treatment, a user 302 can calibrate an initial dose using the dose controller. For example, in some embodiments, the dose controller can calculate an initial treatment based on an approximation of the user's Fitzpatrick skin type (“FST”) classification. The FST classification is correlated to MED and skin color. Increased melanin provides photoprotection, decreasing sun sensitivity and directly correlating with higher UV radiation dosage requirements to produce erythema. An FST self-assessment test can be used to predict an individual's photosensitivity, placing the individual into one of six graduated categories (skin type I-VI). However, this phototype classification is based on responses to a series of questions (e.g., posed by a user interface 142 (FIG. 1A) to the user), which imposes some subjectivity that can cause a higher error index in comparison to objective MED data. In a very simplified version, for example, self-assessment of FST can be done by selecting a skin type from Table 1 that best describes sunburn and tanning history. Data shows that there is a stepwise increase in the average MED from skin types I through VI, but although skin type and MED are correlated, there is a very wide range of MED values within each skin type and a substantial degree of overlap in the MED values among different skin types. Therefore, skin type alone may provide an indication of starting UV radiation dose range based on mean MED, but cannot serve as an absolute predictor of an individual patient's sensitivity to UV light. Other characteristics, such as hair color, skin color, eye color, number of freckles, sunburn propensity and suntan propensity have been tested and found similar or less predictive of MED than the original FST classification method. Historic tanning ability, sunburn susceptibility and untanned skin complexion have been shown to be more reliable predictors of MED. However, basing UV therapy dosage on questionnaires alone would result in sub-optimal treatment due to underdosing or overdosing many patients whose photosensitivity lies above or below the population mean. That is why, in some embodiments, given the mean and standard distribution of MED for each skin type, adjustments can be made by the dose controller or by individual users based on erythema response to the starting dose for a given skin type. More details on FST classification and related methods can be found in International Patent Application Publication No. WO2019/118777, incorporated by reference in its entirety.

TABLE 1
Fitzpatrick Skin Type Self-Assessment

| Skin Type | Sunburn/Tanning History |
|---|---|
| I | Always burns, never tans; sensitive (“Celtic”) |
| II | Burns easily, tans rarely |
| III | Burns moderately, tans gradually to light brown |
| IV | Burns minimally, always tans well to |
| | moderately brown (olive skin) |
| V | Rarely burns, tans profusely to dark brown |
| | (brown skin color) |
| VI | Never burns, deeply pigmented; not sensitive |
| | (black skin |

Fitzpatrick, T.B., Arch. Dermatol., 124, 869, 1988.

In various embodiments, the dose controller can approximate the user's FST using the classic FST classification method; using the user's response to certain questions about their skin tone, hair color, eye color number of freckles, sunburn propensity, and/or suntan propensity; and/or using various other sources of information, such as biographic information, images of the user, etc.

In some embodiments, the user interface 142 on the PED 140 (FIGS. 1A and 1B) can present questions related to the user's skin tone visually with color swatches and/or facial photographs of archetypical characteristics that normally are associated with the skin type categories. FIG. 4 is an illustration of a Fitzpatrick skin tone selection GUI 401 in accordance with embodiments of the present technology. As shown in FIG. 4, people with skin type 1 (most sensitive) usually have light eyes (blue or green) and hair (blond or red), frequently have many freckles, and pinkish pale skin. On the other end of the spectrum, people with skin type 6 normally have very dark eyes (black or dark brown) and hair (black or dark brown), no freckles, and dark brown or black skin. Choosing photographs with these characteristics can increase accuracy of a single skin tone question by allowing users to self-categorize other related characteristics. Using photographs can allow this single question to be used to establish skin type category without other questions. In some embodiments, this question can have photographs without skin tone swatches. In other embodiments, skin tone answers can be described verbally through a speaker or with written description, of untanned skin town such as pinkish pale, pale, moderate pale, moderate dark, dark, or very dark.

In some embodiments, the user interface 142 can present questions related to hair color to assess skin type and photosensitivity. Because skin pigmentation is directly correlated hair pigmentation, a question related to hair color is designed to estimate melanin concentration in the skin from darkness of natural hair color. In some embodiments, examples (e.g., photos or graphics) of a spectrum of hair color from light to dark can be presented on the user interface 142 with or without written description. In some embodiments, only written (or auditory) description is used. The hair color question(s) can be asked in different ways and have a range of answers. For example, the question(s) may include one or more of the following: what is your natural hair color, what was the color of your natural scalp hair as a teenager, and/or how dark was your natural scalp hair as a teenager? The answers provided to the user via the user interface 142 may include the following: very light or red-light blond, light or blond-light brown, moderate or dark blond-brown, medium dark or brown-dark brown, dark or dark brown-black, and/or very dark or black.

In some embodiments, the user interface 142 can present questions related to eye color to assess skin type and photosensitivity. Because skin pigmentation is directly correlated eye pigmentation, the eye color question is designed to estimate melanin concentration in the skin from darkness of natural eye color. In some embodiments, examples (e.g., photos or graphics) of a spectrum of eye color from light to dark can be presented on the user interface 142 with or without written description. In some embodiments, only written and/or auditory descriptions are used. The eye color question can be asked to determine eye lightness or color and have a range of answers. For example, the eye color question(s) may include: what is your natural eye color, or how dark is your natural eye color? The answers provided to the user via the user interface 142 may include the following: very light or light blue-light green, light or blue-green-light hazel, moderate or dark blue-dark green-hazel-light brown, medium dark or dark hazel-brown, dark or dark brown-black, and very dark or black.

In some embodiments, the user interface 142 can present questions related to freckles to assess skin type and photosensitivity. Having freckles at a young age or having many freckles is correlated to a lower minimal erythema dose (MED). Thus, freckle-related questions are designed to distinguish lighter skin types from darker ones by estimating number of freckles. In some embodiments, examples (e.g., photos or graphics) of a patch of skin with a spectrum of different freckle concentrations from many to none can be presented to the user via the user interface 142 with or without written description. In some embodiments, only written or auditory descriptions are used. The freckle-related question(s) can be asked with a yes or no response and/or with a range of answers. For example, the freckle-related question(s) may include: did you have freckles at 10 years old, how many freckles do you have on your body, and/or what percentage of your body skin contains freckles? The answers provided to the user via the user interface 142 may include the following: many or 75-100%, several or 50-75%, some or 25-50%, few or 1-25%, and none or 0%.

In some embodiments, the user interface 142 can present questions related to the user's propensity to sunburn to user's skin type and photosensitivity. Self-reported sunburn propensity is correlated to MED. The sunburn-related question(s) can be asked in different ways and have a range of answers. For example, sunburn-related questions may include: how easily do you sunburn in midday summer sun without sunscreen, and how easily do you sunburn? The answers may include: very easily, easily, moderately, minimally, rarely, and never.

In some embodiments, the user interface 142 can present questions related to the user's propensity to suntan to approximate the user's skin type and photosensitivity. Self-reported suntan potential is correlated to MED. The suntan-related question(s) can be asked in different ways and have a range of answers. For example, suitable suntan-related questions include: how easily does your skin suntan, how tan can you get after one week of daily summer sun, or how long does it take for you to build a good suntan in summer sunlight? Suitable answers to address these questions may include: (a) never, none, very long—never; (b) minimally, light, 10+ days; (c) moderately, moderate, or 5-9 days; (d) easily, dark, or 3-4 days; (e) very easily, very dark, or 1-2 days; and (f) difficult to notice.

The phototherapy system 100 can receive answers via the user interface 142 and/or other device to the questions related to the user's FST, skin tone, hair color, eye color, freckle, propensity to sunburn, propensity to suntan, and/or other photosensitive-related inquiries, and the processor can use these answers to questions to automatically prescribe the user's skin type, the starting/baseline dose, and/or MED prior to phototherapy treatment. The received answers can also be used to analyze against treatment response to create algorithms that can better predict MED using machine learning.

Each answer can be weighted in correlation with skin types one through six. In some embodiments, such as many of the examples described above, the answer sets are provided on a six-point scale designed to correspond with the six skin types (e.g., two points would be assigned to the second answer and correspond to skin type two characteristics). However, answer sets include fewer than six answers, and be divided evenly between six skin types (e.g., answers 1-5 are scored as 1, 2.25, 3.5, 4.75, and 6, respectively). Some answers can be weighted differently so that some characteristics provide a stronger or weaker influence on the overall scoring of multiple questions (e.g., answers 1-6 can be scored as 1, 1.5, 2.5, 3.5, 6, and 8, respectively). Some entire questions can be weighted differently so that they provide a stronger or weaker influence on the overall scoring of multiple questions. For example, the answer to a question can be multiplied by 0.5 to provide half the influence on the overall score or multiplied by two to impart twice the influence on the overall score as normal baseline scored questions. Some questions can be combined with a conditional statement that creates a single answer (or point value) that is not a summation of the questions separately. Answers to questions can be scored to provide a range of point totals that are placed into one of six skin type buckets (e.g., the Fitzpatrick system) that can provide six MED estimates and six starting dosages that correspond to the six skin types. In other embodiments, the scoring of answers can provide a higher resolution skin type, such as 1-100 or 1.0-6.0 with an equally high resolution of MED estimates and starting dosages. The following are some examples of scoring formulas that lead to a skin type (ST) value:

1. Two questions (Q1, Q4) with logic to determine ST 1 to 6:
a. If Q1=A1 then ST=1
b. Else if Q1=A2 then ST=2
c. Else if Q1=A3 then ST=3
d. Else if Q1=A4 then
   i. If Q4<=4 ST=4
   ii. If Q4=5 ST=5
   iii. If Q4=6 ST=6

2. Two questions (Q1, Q4) with logic to determine ST 1 to 6:
a. If Q1=A1 then ST=1
b. Else if Q1=A2 or A3 then
   i. If Q4 value<Q1 value then ST=Q4 value
   ii. Else ST=Q1 value
c. Else if Q1=A4 then ST=Q4 answer (A1-A6)
   i. If Q4=1 then ST=1
   ii. If Q4=2 then ST=2
   iii. If Q4=3 then ST=3
   iv. If Q4=4 then ST=4
   v. If Q4=5 then ST=5
   vi. If Q4=6 then ST=6

3. Single question (Q4) scoring based on skin color pictures and swatches with each answer having the same point value as the answer number (i.e. A4=4 points).
a. ST=Q4 Answer (1-6)

4. Multiple questions with equal weight scoring rounded to the nearest integer (ST 1-6) or decimal (i.e. 2.5 instead of 2 or 3) with each answer having the same point value as the answer number, except Q7 where A1=1 point and A2=4 points.
a. ST=(Q1 value+Q4 value)/2
b. ST=(Q2 value+Q3 value)/2
c. ST=(Q9 value+Q10 value)/2
d. ST=(Q2 value+Q3 value+Q4 value)/3
e. ST=(Q4 value+Q9 value+Q10 value)/3
f. ST=(Q1 value+Q4 value+Q7 value)/3
g. ST=(Q1 value+Q4 value+Q8 value)/3
h. ST=(Q4 value+Q7 value+Q9 value+Q10 value)/4
i. ST=(Q4 value+Q8 value+Q9 value+Q10 value)/4
j. ST=(Q4 value+Q5 value+Q6 value+Q7 value+Q9 value+Q10 value)/6
k. ST=(Q4 value+Q5 value+Q6 value+Q8 value+Q9 value+Q10 value)/6
l. ST=(Q2 value+Q3 value+Q4 value+Q5 value+Q6 value+Q7 value)/6
m. ST=(Q2 value+Q3 value+Q4 value+Q5 value+Q6 value+Q8 value)/6

5. Multiple questions with scoring rounded to the nearest integer (ST 1-6) or decimal and at least one question that conditionally adjusts the formula by increasing/reducing the number of questions for scoring.
a. If Q7=A1 then ST=(Q1 value+Q4 value+1)/3
   i. Else ST=(Q1 value+Q4 value)/2
b. If Q7=A1 then ST=(Q2 value+Q3 value+Q4 value+1)/4
   i. Else ST=(Q2 value+Q3 value+Q4 value)/3
c. If Q7=A1 then ST=(Q4 value+Q9 value+Q10 value+1)/4
   i. Else ST=(Q4 value+Q9 value+Q10 value)/3
d. If Q4=A6 then ST=(Q4 value+Q9 value)/2 or ST=(Q4 value+Q2 value)/2
   i. Else if Q4=A5 then ST=(Q4 value+Q9 value)/2 or ST=(Q4 value+Q2 value)/2
   ii. Else ST=(Q4 value+Q9 value+Q10 value)/3 or ST=(Q4 value+Q2 value+Q3 value)/3
e. If Q4=A6 and Q7=A1 then ST=(Q4 value+Q9 value+1)/3 or ST=(Q4 value+Q2 value+1)/3
   i. Else if Q4=A6 and Q7=A1 then ST=(Q4 value+Q9 value+1)/3 or ST=(Q4 value+Q2 value+1)/3
   ii. Else ST=(Q4 value+Q9 value+Q10 value)/3 or ST=(Q4 value+Q2 value+Q3 value)/3

In some embodiments, the user interface 142 can request one or more photographs from the user to be used in determining the user's skin type. The one or more photographs can then be run through an algorithm to determine the user's likely skin type. In some embodiments, for example, the algorithm can be a machine learning algorithm trained to recognize skin type indicators in photographs to implement the method disclosed above. In some embodiments, the algorithm can be a machine learning algorithm trained to use its own indicators to determine a user's skin type.

Once the user's Fitzpatrick skin type is approximated, the dose controller can work with a user to initiate a calibration process to determine a safe dose threshold for the user's skin. In the calibration process, the dose controller will gradually increase the user's dosage over a number of treatments to gradually determine the dose time to achieve the desired MED. For example, in some embodiments, the desired MED can be set to avoid pigmentary changes in the user's skin lasting more than 6 days, which has been shown to occur for exposures in excess of about 1 MED. Accordingly, in some embodiments, the dose controller can follow the calibration process to set a dose to deliver between about 0.5 MED and 0.7 MED, or about 0.6 MED to the user.

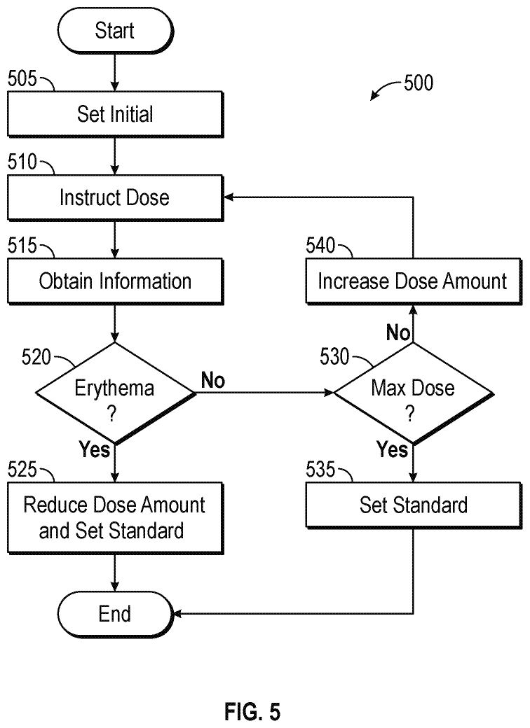

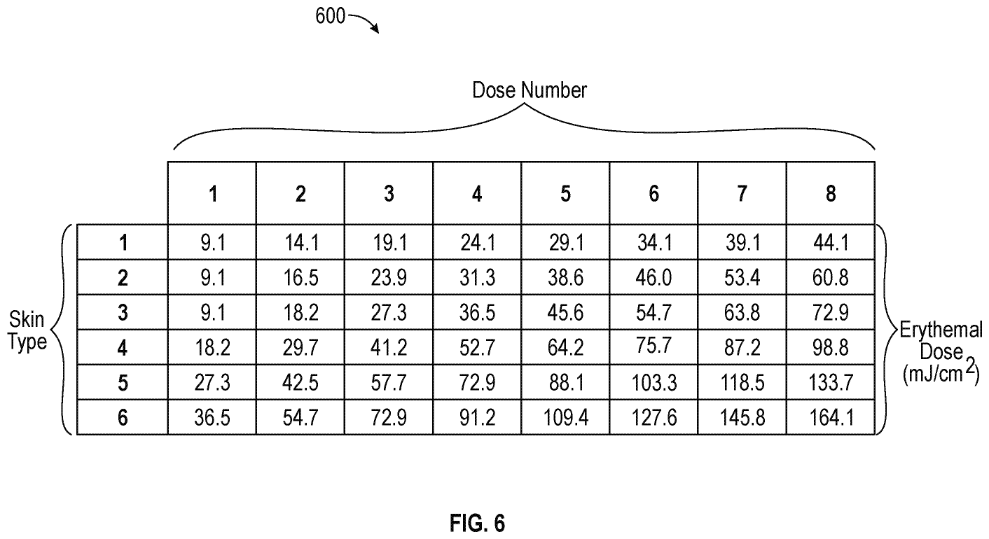

The calibration process in accordance with some embodiments of the present technology is described in detail below with reference to FIGS. 5 and 6. FIG. 5 is a flow diagram illustrating a process 500 for calibrating a dose of UV emissions for a user in accordance with some embodiments of the present technology. FIG. 6 illustrates a dosage chart 600 for various skin types with increasing dosages and expected erythemal doses at varying stages of the calibration process of FIG. 5 in accordance with some embodiments of the present technology.

At block 505, the dose controller sets an initial dosage, based on the skin type determined above. For example, the first row of the dosage chart 600 in FIG. 6 illustrates the dosages for a user with skin type I. At block 505, the dose controller would set the initial dosage to about 9.1 millijoules per square centimeter (mJ/cm²). In some embodiments, the initial dosage can take about 30 seconds to deliver. At block 510, the dose controller can provide the dose to the user. For example, at block 510, the dose controller can communicate with the UV light assembly to begin and end emitting phototherapeutic UV radiation.

At block 515, the dose controller can receive inputs from the user to obtain information regarding the user's reaction to previous dose. In some embodiments, the dose controller can present the user with a series of questions to determine whether the user experienced erythema and/or any other side effects. For example, the dose controller can present a series of questions through the user interface 142 (FIG. 1A). In some embodiments, the dose controller can communicate with either the data gathering component 128 or the PED to request one or more photos of the treated skin.

At decision block 520, the dose controller can decide whether harmful erythema occurred using the information obtained at block 515. If erythema occurred, the dose controller can move to block 525 to set the dosage at a lower threshold and complete the calibration process 500; else the dose controller can move to decision block 530. In some embodiments, if erythema occurs after the initial dose, the dose controller can re-evaluate the user's assigned skin type and restart the calibration process.

At decision block 530, the dose controller can determine whether a maximum dosage has been reached. If a maximum has been reached, the dose controller can move to block 535 to set the dosage at the maximum and complete the calibration process 500; else the dose controller can move to block 540.

At block 540 the dose controller can increase the dosage by one phase and return to block 510 to repeat. For example, with reference to the second row of the table in FIG. 6, the next dosage can be set to deliver a dose of 16.5 mJ/cm², with a corresponding second dose duration dependent on the output of the UV-emitting device. Blocks 510-540 can then be repeated until they reach an end, corresponding to an appropriate dosage for the user.

As illustrated in the charts of FIG. 6, in some embodiments, the calibration process 500 can take between 1-8 doses before the user's normal dosage is set. In some embodiments, the dose controller can restart the calibration process 500 if the user misses one or more doses to reduce the risk of over exposure. In some embodiments, the dose controller can restart and/or re-engage the calibration process 500 in response to an indication from the user that erythema occurred at the set dosage.

In various embodiments, depending on the size of the UV-emitting device 110 (FIG. 1A), the intensity of the UV light emitters 116 used therein, and/or a desired dose the time required to deliver the desired dose can range between about 30 seconds and about 20 minutes, or between about 1 minute and about 15 minutes.

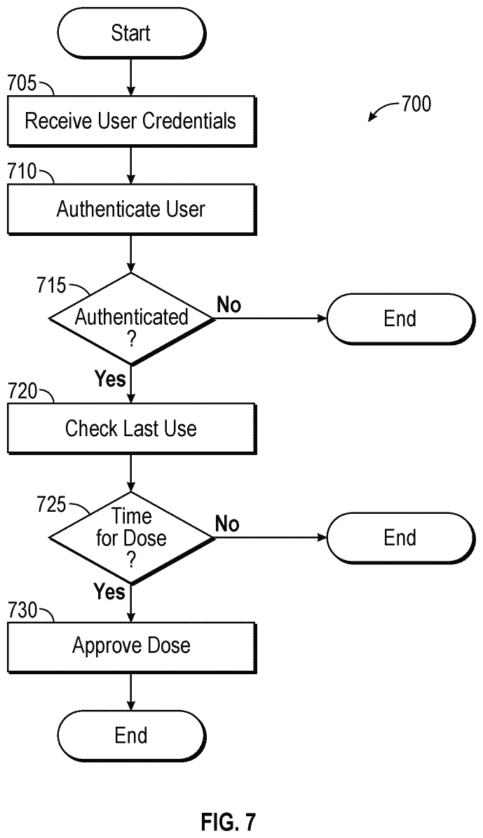

FIG. 7 is a flow diagram of an authentication process 700 (“process 700”) for a dynamic dosing system configured in accordance with some embodiments of the present technology. The authentication protocol can reduce the chance that someone is unknowingly exposed to the phototherapeutic UV radiation, which could cause damage to the person's skin. Purely by way of example, implementing the authentication protocol can reduce the chance a child is exposed to radiation from the system without understanding what light they are turning on.

At block 705, the dose controller can receive input credentials from a user. In some embodiments, the input credentials can be a user name and password, for example received through the user interface 142 on the PED 140. In some embodiments, the input credentials can be a personal identification number (PIN) used as a shortcut to identify the user. In some embodiments, the input credentials can be biometric information from the user, such as a finger print, hand print, facial image, etc. received through the data gathering component 128, the PED 140, another input means, and/or any combination therein.

At block 710, the dose controller authenticates the user. For example, in some embodiments, the dose controller compares the input credentials against the input credentials for users in a registered user list.

At decision block 715, the dose controller determines whether to proceed based on whether the user was authenticated. If the user was authenticated, the dose controller proceeds to block 720; else the dose controller ends the process 700. In some embodiments, the dose controller can transmit a failure message before ending with an indication of the reason for the failure (e.g., password incorrect, no match for input credentials, etc.).

At block 720, the dose controller checks the last recorded use of the system 100 by the authenticated user. The last-use check can ensure that the authenticated user is not returning for a dose ahead of schedule. For example, in some embodiments, the system can be configured to be used every three to four days. If the user attempts to receive a dose after only one or two days, the dose controller can detect the over-use at decision block 725 using the information retrieved at block 720.

At decision block 725, if the dose controller determines it is time for the user's dose, the dose controller continues to block 730; else the dose controller ends the process 700. In some embodiments, the dose controller can transmit a failure message before ending with an indication of the reason for the failure (e.g., not time for next dose, insufficient recovery period, etc.).

At block 730, the dose controller approves the dose of phototherapeutic UV radiation. In some embodiments, the approval includes transmitting an indication of the approval to the user (e.g., transmitting a message to the user interface 142). In some embodiments, the approval includes transmitting a signal to power the UV light assembly 114 on for the duration of the period for delivering the dose. In some embodiments, the approval includes recording data on the dose for use in the next authentication protocol.

Further Embodiments of the UV Light Assembly

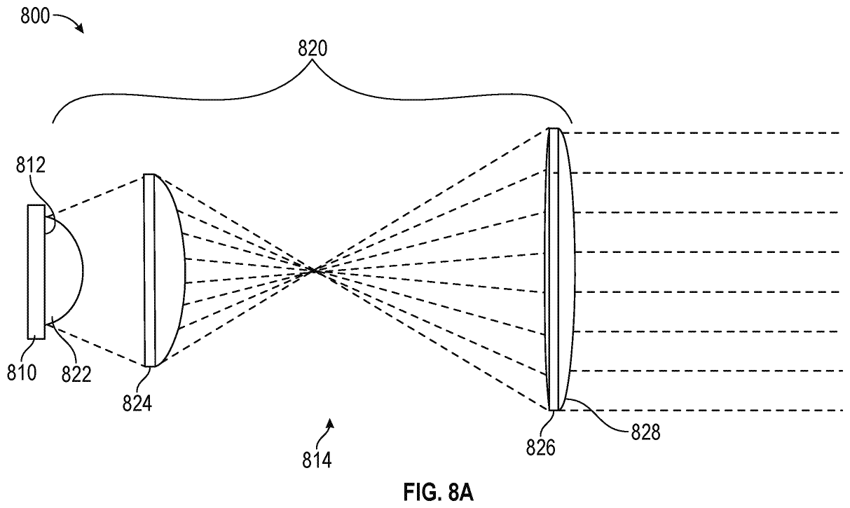

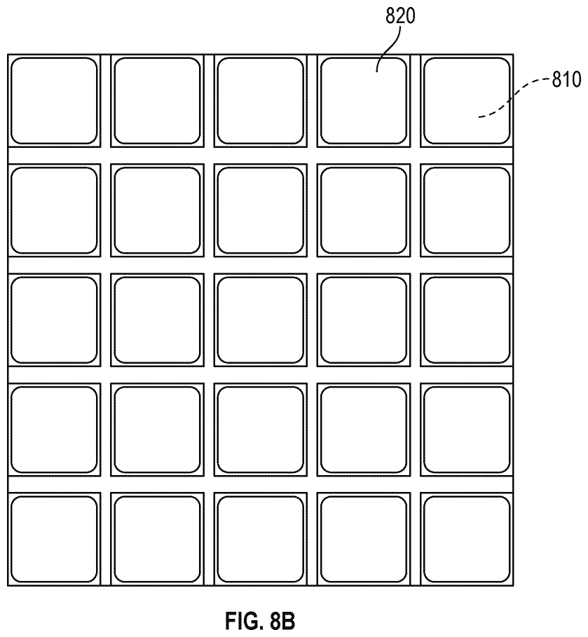

FIGS. 8A-10B illustrate a few additional embodiments of the components of the UV light assembly 114 (FIG. 1A). However, the present technology is not limited to only those embodiments illustrated herein. Rather, one of skill in the art will understand that various other UV radiation sources can be used in conjunction with the system 100 without departing from the scope of the present technology. Purely by way of example, a film of microplasma configured to emit UV radiation in the spectrum disclosed above may also be used as a component of the UV light assembly 114, as noted above.

FIG. 8A is a side view of a component 800 in a UV light assembly 114 configured in accordance with some embodiments of the present technology. In the illustrated embodiment, the component 800 includes an LED 810 configured to emit UV radiation from an active side 812 and an optical element 820 coupled to the LED 810. The optical element 820 includes a first lens 822 attached to the active side 812 of the LED 810, a second lens 824 spaced apart from the first lens 822, a collimating lens 826 spaced apart from the second lens 824, and a protective cover 828 attached to the collimating lens 826.

As illustrated in FIG. 8A, the first lens 822 and second lens 824 are configured to focus the UV radiation 814 emitted by the LED 810 on the collimating lens 826. The collimating lens 826 is configured to direct the UV radiation 814 into a travel path generally orthogonal to the active surface of the LED 810 towards the phototherapy zone 320 (FIG. 3). Together, the first and second lens 822, 824 can function to improve the percentage of UV radiation 814 emitted that is incident on the collimating lens 826, thereby improving the percentage of UV radiation 814 directed the phototherapy zone 320. Further, the optical element 820 can improve the uniformity of average irradiance in (and/or distribution of) the UV radiation 814 leaving the system 100.

The first and second lenses 822, 824 and the collimating lens 826 can be made from various suitable UV resistant materials. For example, in some embodiments, the first and second lenses 822, 824 can be fused silica lenses. In some embodiments, the collimating lens 826 can be a glass lens made from Kopp 9531. In some embodiments, the collimating lens 826 can have a center thickness of 25 millimeters and a diameter of about 3.6″.

FIG. 8B is a top view of a section of the UV light assembly 114 of FIG. 8A configured in accordance with some embodiments of the present technology. In the illustrated embodiment, the section of the UV light assembly 114 includes a five-by-five (5×5) array of LEDs 810 connected to a five-by-five (5×5) array of optical elements 820. In some embodiments, the UV light assembly 114 can be manufactured in five-by-five sections that are then interconnected to form the complete UV light assembly 114 to improve manufacturing speeds and convenience. In various other embodiments, the UV light assembly 114 can be manufactured in various other sized arrays ranging from a single component 800 to the entire assembly altogether. In some embodiments, the components 800 can be manufactured with a gap of about 0.11″ between each other in the array. Accordingly, in some embodiments the array illustrated in FIG. 8B can have dimensions of about 18.55″ by about 18.55″.

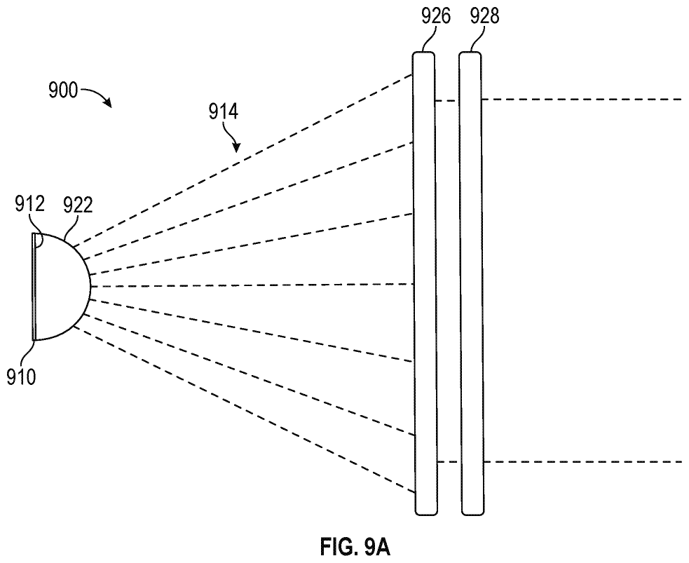

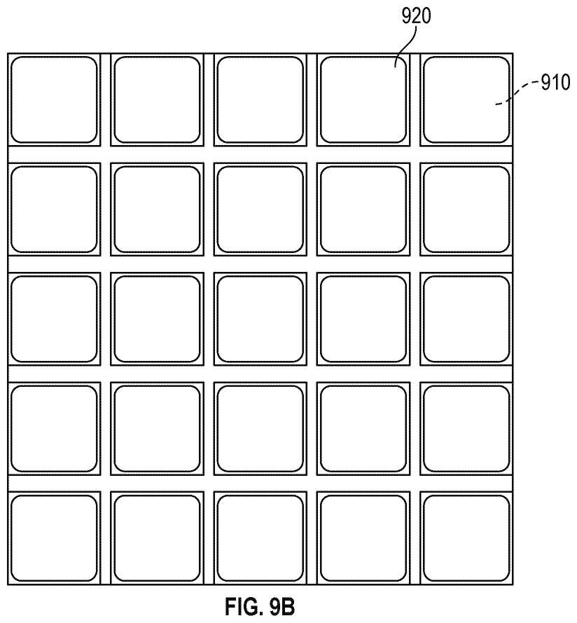

FIG. 9A is a side view of a component 900 in a UV light assembly 114 configured in accordance with some embodiments of the present technology. In the illustrated embodiment, the component 900 includes an LED 910 configured to emit UV radiation 914 from an active side 912 and an optical element 920 coupled to the LED 910. The optical element 920 includes a lens 922 attached to the entire active side 912 of the LED 910, a collimating lens 926 spaced apart from the lens 922, and a filter 928 attached to the collimating lens 926.

As illustrated in FIG. 9A, the first lens 822 is configured to direct and/or disperse the UV radiation 914 emitted by the LED 910 towards the collimating lens 826. The collimating lens 926 is configured to direct the UV radiation 914 into a travel path generally orthogonal to the active side 912 and towards the phototherapy zone 320 (FIG. 3). The lens 922 can improve the percentage of UV radiation 914 emitted that is incident on the collimating lens 926, thereby improving the percentage of UV radiation 914 directed the phototherapy zone 320. Further, taken together, the optical element 920 can improve the uniformity of average irradiance in (and/or distribution of) the UV radiation 914 leaving the system 100.

In some embodiments, the lens 922 can be a fused silica lens. In some embodiments, the collimating lens 926 can be a Fresnel pattern collimating lens. In some embodiments, the collimating lens 926 can made from glass; in other embodiments, the collimating lens can be made from plastic. In some embodiments, the collimating lens 926 can have a diameter of about 3.6″. In some embodiments, the filter 928 can block UV radiation 914 outside of the desired spectrum from leaving the system 100. In some embodiments, the filter 928 can operate as a protective cover to the optical element 920.

FIG. 9B is a top view of a section of the UV light assembly 114 of FIG. 9A configured in accordance with some embodiments of the present technology. In the illustrated embodiment, the section of the UV light assembly 114 includes a five-by-five (5×5) array of LEDs 910 connected to a five-by-five (5×5) array of optical elements 920. In some embodiments, the UV light assembly 114 can be manufactured in five-by-five sections that are then interconnected to form the complete UV light assembly 114 to improve manufacturing speeds and convenience. In various other embodiments, the UV light assembly 114 can be manufactured in various other sized arrays ranging from a single component 900 to the entire assembly altogether. In some embodiments, the components 900 can be manufactured with a gap of about 0.11″ between each other in the array. Accordingly, in some embodiments the array illustrated in FIG. 9B can have dimensions of about 18.55″ by about 18.55″.

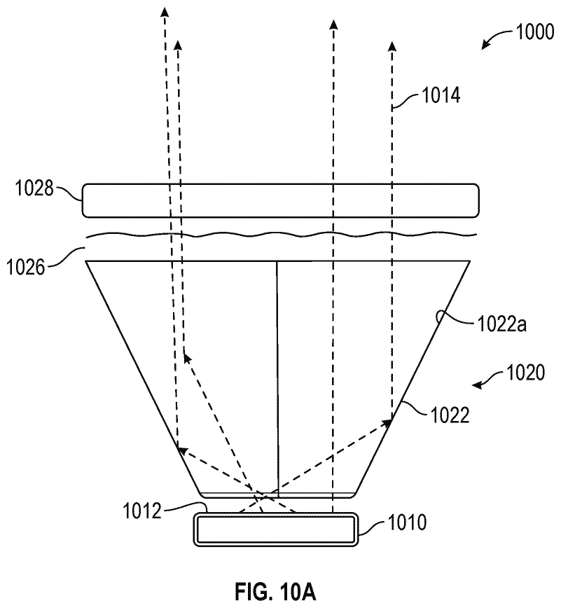

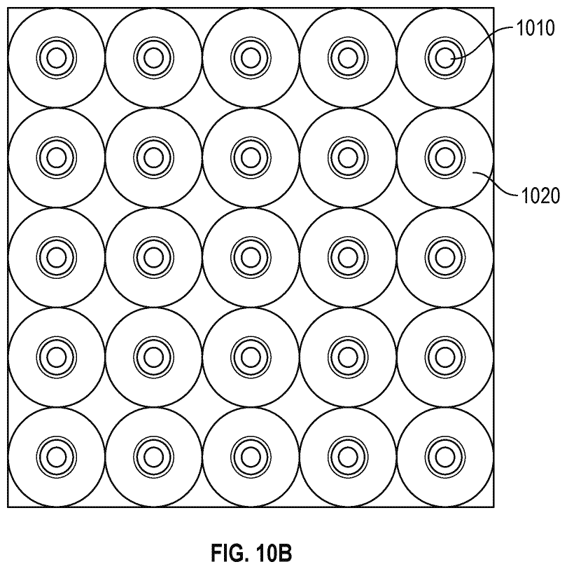

FIG. 10A is a side view of a component 1000 in a UV light assembly 114 configured in accordance with some embodiments of the present technology. In the illustrated embodiment, the component 1000 includes an LED 1010 configured to emit UV radiation 1014 from an active side 1012 and an optical element 1020 coupled to the LED 1010. The optical element 1020 includes a total internal reflection lens 1022 (“TIR lens 1022”) attached to the entire active side 1012 of the LED 1010, a collimating lens 1026 attached to the TIR lens 1022, and a filter 1028 attached to the collimating lens 1026.

The TIR lens 1022 can be made from one or more suitable high UVB transmissive materials. For example, in some embodiments, the TIR lens 1022 is made from a high transparency liquid silicone rubber (e.g., Silopren LSR 7000 or 7080J series rubber). In some embodiments, the TIR lens 1022 is made from a UV-resistant polymer (e.g., an Acrypet® resin or other suitable UV-resistant polymer). When made from a polymer-based material, the TIR lens 1022 can be thinner and lighter than a silicon-based lens, without compromising the UV-durability of the lens material. In some embodiments, the collimating lens 1026 can be a Fresnel pattern collimating lens. In some embodiments, the collimating lens 1026 can be made from glass; in other embodiments, the collimating lens can be made from plastic. In some embodiments, the filter 1028 can block UV radiation 1014 outside of the desired spectrum from leaving the system 100. In some embodiments, the filter 1028 can operate as a protective cover to the optical element 1020.

FIG. 10B is a top view of a section of the UV light assembly 114 of FIG. 10A configured in accordance with some embodiments of the present technology. In the illustrated embodiment, the section of the UV light assembly 114 includes a five-by-five (5×5) array of LEDs 1010 connected to a five-by-five (5×5) array of optical elements 1020. In some embodiments, the UV light assembly 114 can be manufactured in five-by-five sections that are then interconnected to form the complete UV light assembly 114 to improve manufacturing speeds and convenience. In various other embodiments, the UV light assembly 114 can be manufactured in various other sized arrays ranging from a single component 1000 (FIG. 10A) to the entire assembly altogether.

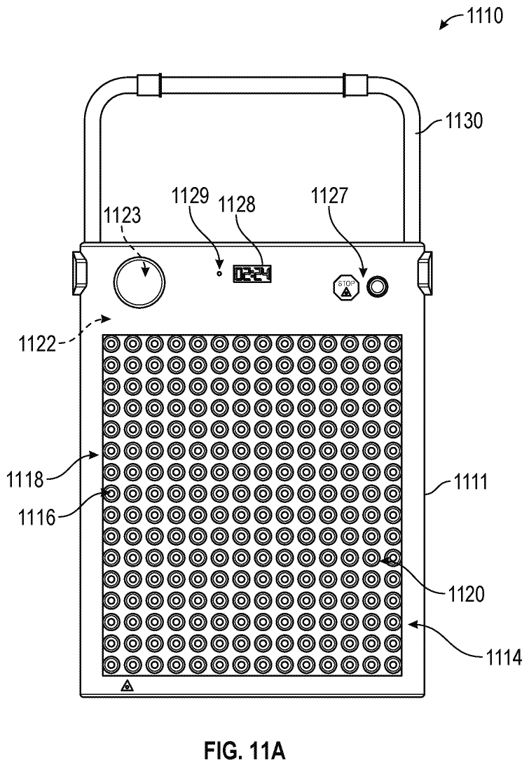

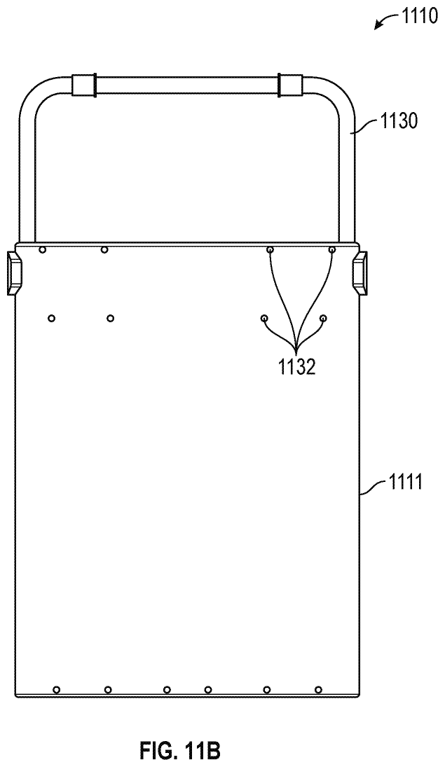

FIGS. 11A and 11B are a front and rear view, respectively, of a UV-emitting device 1110 for a portable phototherapy system in accordance with some embodiments of the present technology. As illustrated in FIG. 11A, the UV-emitting device 1110 includes a housing 1111 and a UV light assembly 1114 carried by the housing 1111. In the illustrated embodiment, the UV light assembly 1114 includes an array of UV light emitters 1116 (e.g., LEDs) and an optical component 1118 that includes an array of TIR lenses 1120, with each individual TIR lens generally corresponding to an individual UV light emitter. Accordingly, when the UV-emitting device 1110 is powered on, the UV-emitting device 1110 emits UV radiation away from the active surface 1112a of the housing 1111 in a generally uniform manner. In various embodiments, the array of TIR lenses 1120 can be constructed from any suitable UV-resistant polymer resin and/or any suitable UV-resistant rubber. In some embodiments, the optical component 1118 includes an array of reflectors and/or another optical lens instead of (or in addition to) the array of TIR lenses 1120.

In the embodiment illustrated in FIG. 11A, the UV-emitting device 1110 also includes an electronic component 1122 contained within the housing 1111. The electronic component 1122 can control the operation of the UV-emitting device 1110. For example, in various embodiments, the electronic component can control the power supply to the UV light assembly 1114, communicate with other electronic devices (e.g., the PED 140 of FIG. 1A), implement the dose-defining protocol and/or authentication protocol, and/or implement various other safety features. To facilitate some of these features, the internal electronic component 1122 can be operably coupled to a communication component 1123, an emergency stop button 1127, a display 1128, and a proximity sensor 1129.

The communication component 1123 can include a short-range wireless component and/or a network communication component allowing the internal electronic component 1122 to communicate with a PED 140 and/or a cloud server 150 (FIG. 1B). The communication component 1123 can also include a radio-frequency identification (RFID) reader. The RFID reader can help ensure that the user has a relevant safety feature with them before the electronic component 1122 provides power to the UV light assembly 1114. For example, the electronic component 1122 can require that the RFID reader be presented with an RFID tag on safety glasses before the electronic component 1122 provides power to the UV light assembly 1114. By ensuring the user at least has the relevant safety feature with them, the electronic component 1122 is expected to improve the safety of using the UV-emitting device 1110.

As illustrated in FIG. 11A, the emergency stop button 1127 can be prominently displayed and easily accessed on the housing 1111 of the UV-emitting device 1110. The emergency stop button 1127 allows a user (or another person) to quickly power off the UV-emitting device 1110 in the case of an accidental exposure, an experienced burn from an overexposure, and/or any other urgent situation. In some embodiments, the electronic component 1122 can record the time of stopping to measure the dose of UV radiation that was delivered before the emergency stop button 1127 was pressed. The measurement can then be communicated to a PED 140 and/or a cloud server 150 (FIG. 1B) for review by the user, a medical care provider, and/or the dose controller. For example, the user can present the record to their doctor when seeking treatment for a burn to help communicate the severity of the burn and/or explain any symptoms. In another example, the dose controller can use the record when setting and/or adjusting a next dose. In a particular example, the user can press the emergency stop button 1127 when their child enters the room to avoid unintentional exposure to the UV radiation, then seek to complete their dose of UV radiation after they take their child out of the room.

The display 1128 can be used to communicate information to and/or receive information from the user. For example, as illustrated in FIG. 11A, the display 1128 can include an indication of the time remaining on a dose of UV radiation. In some embodiments, the display also includes an indication of the time elapsed, the dose that has already delivered, and/or any other suitable information. Before the UV light assembly 1114 is powered on, the display 1128 can include instructions to the user for using the UV-emitting device 1110. Further, like the data gathering component 128 discussed above with reference to FIG. 1A, the display can be a touchscreen display that receives inputs from the user. Purely by way of example, the display 1128 can receive inputs related to a user authentication before the electronic component 1122 provides power to the UV light assembly 1114.

The proximity sensor 1129 can help ensure proper usage of the UV-emitting device 1110 by detecting how far away the user is standing while receiving a dose of UV-radiation. For example, the electronic component 1122 can use information from the proximity sensor 1129 to determine whether the user is standing within the phototherapy zone 320 (FIG. 3). If not, the UV-emitting device 1110 can provide audio/visual feedback to the user to instruct them to adjust their position. For example, when the user stands too close to the UV-emitting device 1110 (e.g., less than the first distance X₁ discussed above with respect to FIG. 3), the UV-emitting device 1110 can provide an audible indication that they are too close. Similarly, when the user stands too far to the UV-emitting device 1110 (e.g., more than the second distance X₂ discussed above with respect to FIG. 3), the UV-emitting device 1110 can provide an audible indication that they are too far away. The audio/visual feedback can prompt the user to adjust their position, thereby increasing the amount of time that they are within the phototherapy zone 320 (FIG. 3) and receive an appropriate dose of UV radiation. In some embodiments, when the user stands too close to the UV-emitting device 1110 for too long (e.g., for 5 seconds, 10 seconds, 20 seconds, or any other suitable time period), the electronic component 1122 cuts off the power from the UV light assembly 1114. As discussed above, the UV radiation gradually disperses as it travels away from a UV-emitting device. Therefore, the UV radiation is more concentrated (and more intense) immediately adjacent the UV-emitting device 1110. Accordingly, the cut-off enforced by the electronic component 1122 can help prevent the user from accidentally receiving too high (or too intense) of a dose of the UV radiation.

As further illustrated in FIGS. 11A and 11B, the UV-emitting device 1110 can include a hang-bar 1130 that allows the user to hang the UV-emitting device 1110 in a suitable location (e.g., on the back of a door). The hang-bar 1130 can have an adjustable height that allows the elevation of the UV-emitting device 1110 (and therefore the UV light assembly 1114) to be tailored to the user. In some embodiments, the adjustable height is achieved through telescoping components on the hang-bar 1130. In some embodiments, the adjustable height is achieved through internal tracks in the housing 1111, allowing the hang-bar 1130 to be pushed into and/or pulled out of the housing 1111.

As illustrated in FIG. 11B, the UV-emitting device 1110 can also include one or more mounting holes 1132 (four labeled, fourteen illustrated) on the housing 1111. The mounting holes 1132 can allow any other suitable mounting component (e.g., an independent stand, other hanging features, suction features, and the like) to be attached to the UV-emitting device 1110. Accordingly, the mounting holes 1132 allow the user to customize the mounting features based on their desired use for the UV-emitting device 1110. Additionally, or alternatively, the mounting holes 1132 can allow various accessories to be attached to the UV-emitting device 1110.

### EXAMPLES

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A home phototherapy system for producing vitamin D via skin exposure, the home phototherapy system comprising:
a portable UV-emitting device that includes:
   a housing having an active side;
   a UV light assembly within the housing, the UV light assembly including—
      an array of UV light emitters positioned to emit phototherapeutic UV radiation having a peak wavelength between 293 nm and 299 nm away from the active side;
      an optical component disposed on the UV light emitters, the optical component configured to direct the phototherapeutic UV radiation outwardly from the housing to a phototherapy zone; and
a dose controller communicably coupled to the UV light assembly, wherein the dose controller is configured to execute a dose-defining protocol to determine a dosage the phototherapeutic UV radiation to promote vitamin D production via a user's skin.

2. The home phototherapy system of example 1, further comprising an electronic device in communicably coupled between the portable UV-emitting device and the dose controller, wherein the electronic device is configured to receive inputs related to the dose-defining protocol and communicate the inputs to the dose controller.

3. The home phototherapy system of any of examples 1 and 2, further comprising a cloud server communicably coupled to the portable UV-emitting device, wherein the dose controller is implemented on the cloud server.

4. The home phototherapy system of any of examples 1-3 wherein the array of light emitters is an array of light emitting diodes (LEDs) configured to emit UV radiation.

5. The home phototherapy system of example 4 wherein the optical component comprises an array of total internal reflection (TIR) lenses positioned to improve the uniformity of UV radiation emitted from the LEDs towards the phototherapy zone, and wherein each individual TIR lens generally corresponds to an individual LED in the array of LEDs.

6. The home phototherapy system of example 4 wherein the optical component includes an array of optical lenses positioned to collimate UV radiation emitted from the LEDs, and wherein each individual optical lens in the array of optical lenses generally corresponds to an individual LED in the array of LEDs.

7. The home phototherapy system of example 4 wherein the optical component comprises an array of reflectors positioned to improve the uniformity of UV radiation emitted from the LEDs in the phototherapy zone, and wherein each individual reflector generally corresponds to an individual LED in the array of LEDs.

8. The home phototherapy system of any of examples 1-7 wherein the array of UV light emitters comprises a microplasma film having an array of microcavities configured to emit UV radiation.

9. The home phototherapy system of any of examples 1-8 wherein defining the dosing protocol includes:
determining, based on inputs from the user, a skin type associated with the user; and
determining, based on the skin type associated with the user, an initial dosage of the phototherapeutic UV radiation is configured to limit UV exposure to 0.5-0.7 MED based on the skin type associated with the user.

10. The home phototherapy system of any of examples 1-9 wherein defining the dosing protocol includes:
receiving inputs from the user to obtain information related to the user's reaction to a first dosage of the phototherapeutic UV radiation;
determining whether the user experienced erythema;
wherein:
   if the user experienced erythema, determining a dose protocol to deliver a second dosage of the phototherapeutic UV radiation smaller than the first dosage, and
   if the user did not experience erythema, determining a dose protocol to deliver a third dosage of the phototherapeutic UV radiation equal to or greater than the first dosage.

11. The home phototherapy system of any of examples 1-10 wherein the dose controller is further configured to execute an authentication protocol, the authentication protocol including—
receiving input credentials from the user;
authenticating the user using a registered user system;
determining, based at least partially on the result of the user authentication, whether to communicate with the UV-emitting device to power the UV light assembly on;
determining, based at least partially on the user's last access, whether to communicate with the UV-emitting device to power the UV light assembly on; and
indicating to the user whether the UV light assembly will be powered on.

12. The home phototherapy system of any of examples 1-11 wherein the optical component is configured to direct the phototherapeutic UV radiation outwardly toward the phototherapy zone such that the phototherapeutic UV radiation has a uniform irradiance in the phototherapy zone.

13. The home phototherapy system of any of examples 1-11 wherein the phototherapy zone is between a first distance from the active surface of the housing and second distance from the active surface larger than the first distance, and wherein the optical component is configured to direct the phototherapeutic UV radiation outwardly such that the phototherapeutic UV radiation has an irradiance that diverges by less than 10% between the first distance and the second distance.

14. A phototherapy system for producing vitamin D via skin exposure to ultraviolet (UV) radiation, the home phototherapy system comprising:
an ultraviolet-emitting (UV-emitting) device having:
   a housing with an active surface;
   a UV light assembly carried by the housing and positioned to emit positioned to emit phototherapeutic UV radiation having a peak wavelength between 293 nm and 299 nm away from the active surface;
   an optical component disposed over the UV light assembly and configured to collimate the phototherapeutic UV radiation to improve an average distribution of the phototherapeutic UV radiation exiting the UV-emitting device; and
   an electronics controller in operably coupled to the UV light assembly;
an application executable on an electronic device, the application configured to use the electronic device to communicate with the electronics controller to provide a dosing protocol to the electronics controller, wherein the dosing protocol defines a dosage of the phototherapeutic UV radiation for a user; and
a dose controller communicatively coupled to the application, wherein the dose controller is configured to execute a dose-defining protocol to define the dosing protocol and communicate the dosing protocol to the application.

15. The home phototherapy system of example 14 wherein the application is further configured to execute an authentication protocol, the authentication protocol including—
receiving input credentials from the user through the electronic device;
authenticating the user using a registered user system;
determining, based at least partially on the result of the user authentication, whether to communicate with the UV-emitting device to power the UV light assembly on; and
indicating to the user whether the UV light assembly will be powered on.

16. The home phototherapy system of example 15 wherein the authentication protocol further including determining, based at least partially on the user's last access, whether to communicate with the UV-emitting device to power the UV light assembly on.

17. The home phototherapy system of any of examples 14-16 wherein the dose-defining protocol includes:
receiving inputs from the user to obtain information related to the user's reaction to a first dosage of the phototherapeutic UV radiation;
determining whether the user experienced erythema;
wherein:
   if the user experienced erythema, determining a dose protocol to deliver a second dosage of the phototherapeutic UV radiation smaller than the first dosage, and
   if the user did not experience erythema, determining a dose protocol to deliver a third dosage of the phototherapeutic UV radiation equal to or greater than the first dosage.

18. The home phototherapy system of any of examples 14-17 wherein the UV-emitting device further comprises a proximity sensor positioned to detect a distance of the user away from the active surface while the UV light assembly is powered one, and wherein the electronics controller is operably coupled to the proximity sensor to power the UV light assembly off if the distance of the user is below a predetermined threshold for a predetermined period.

19. A method for operating a phototherapy system for producing vitamin D via skin exposure to ultraviolet (UV) radiation, the method comprising:
executing a dose-determining protocol, wherein the dose-determining protocol includes—
   receiving, from a user of the home phototherapy system, inputs related to the user's skin type;
   determining, from the inputs related to the user's skin type, a minimal erythemal dose (MED) associated the user; and
   determining, based on the MED associated the user, a dosing protocol to deliver an initial dosage of phototherapeutic UV radiation for the user; and
sending the dosing protocol to a UV-emitting device in the phototherapy system.

20. The method of example 19 wherein the dose-determining protocol further includes—
receiving, from the user, inputs related to the user's reaction to the initial dosage of the phototherapeutic UV radiation;
determining whether the user experienced erythema;
if the user experienced erythema, determining an updated dose protocol to deliver a second dosage of the phototherapeutic UV radiation smaller than the initial dosage, and
if the user did not experience erythema, determining the updated dose protocol to deliver a third dosage of the phototherapeutic UV radiation greater than the initial dosage.

21. The method of any of examples 19 and 20, further comprising executing a user authentication protocol, wherein the user authentication protocol including—
receiving, from the user, credentials specific to the user;
authenticating the user using a registered user system;
determining, based at least partially on the result of the user authentication, whether to send the dosing protocol to the UV-emitting device; and
providing, to the user, an indication of whether the dosing protocol will be sent to the UV-emitting device.

22. A non-transitory computer-readable storage medium, the computer-readable storage medium including instructions that when executed by a computer, cause the computer to:
execute a dose-determining protocol, wherein the dose-determining protocol includes—
   receiving, from a user of the home phototherapy system, inputs related to a skin type for the user;
   determining, from the inputs related to the user's skin type, a minimal erythemal dose (MED) associated the user; and
   determining, based on the MED associated the user, a dosing protocol to deliver an initial dosage of phototherapeutic UV radiation for the user; and
send the dosing protocol to a UV-emitting device in the phototherapy system.

23. The computer-readable storage medium of example 22 wherein the dose-determining protocol further includes—
receiving, from the user, inputs related to the user's reaction to the initial dosage of the phototherapeutic UV radiation; and
determining whether the user experienced erythema;
wherein:
   if the user experienced erythema, the dose-determining protocol further includes determining an updated dose protocol to deliver a second dosage of the phototherapeutic UV radiation smaller than the initial dosage, and
   if the user did not experience erythema, the dose-determining protocol further includes determining the updated dose protocol to deliver a third dosage of the phototherapeutic UV radiation greater than the initial dosage.

24. The computer-readable storage medium of example 22, wherein the instructions further cause the computer to execute a user authentication protocol, wherein the user authentication protocol includes—
receiving, from the user, credentials specific to the user;
authenticating the user using a registered user system;
determining, based at least partially on the result of the user authentication, whether to send the dosing protocol to the UV-emitting device; and
providing, to the user, an indication of whether the dosing protocol will be sent to the UV-emitting device.

### CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded.

Embodiments of the present disclosure may be implemented as computer-executable instructions, such as routines executed by a general-purpose computer, a personal computer, a server, or other computing system. The present technology can also be embodied in a special purpose computer or data processor that is specifically programmed, configured, or constructed to perform one or more of the computer-executable instructions explained in detail herein. The terms “computer” and “computing device,” as used generally herein, refer to devices that have a processor and non-transitory memory, as well as any data processor or any device capable of communicating with a network. Data processors include programmable general-purpose or special-purpose microprocessors, programmable controllers, ASICs, programming logic devices (PLDs), or the like, or a combination of such devices. Computer-executable instructions may be stored in memory, such as RAM, ROM, flash memory, or the like, or a combination of such components. Computer-executable instructions may also be stored in one or more storage devices, such as magnetic or optical-based disks, flash memory devices, or any other type of non-volatile storage medium or non-transitory medium for data. Computer-executable instructions may include one or more program modules, which include routines, programs, objects, components, data structures, and so on that perform particular tasks or implement particular abstract data types.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

We claim:

1. A home phototherapy system for producing vitamin D via skin exposure to ultraviolet (UV) radiation, the home phototherapy system comprising:
a portable UV-emitting device that includes:
a housing having an active side;
a UV light assembly within the housing, the UV light assembly including—
an array of UV light emitters positioned to emit phototherapeutic UV radiation having a peak wavelength between 293 nm and 299 nm away from the active side;
an optical component disposed on the UV light emitters, the optical component configured to direct the phototherapeutic UV radiation outwardly from the housing to a phototherapy zone a distance away from the active side of the housing; and
a dose controller communicably coupled to the UV light assembly, wherein the dose controller is configured to execute a dose-defining protocol to determine a dosage the phototherapeutic UV radiation to promote vitamin D production via a user's skin.

2. The home phototherapy system of claim 1, further comprising an electronic device in communicably coupled between the portable UV-emitting device and the dose controller, wherein the electronic device is configured to receive inputs related to the dose-defining protocol and communicate the inputs to the dose controller.

3. The home phototherapy system of claim 1, further comprising a cloud server communicably coupled to the portable UV-emitting device, wherein the dose controller is implemented on the cloud server.

4. The home phototherapy system of claim 1 wherein the array of light emitters is an array of light emitting diodes (LEDs) configured to emit UV radiation.

5. The home phototherapy system of claim 4 wherein the optical component comprises an array of total internal reflection (TIR) lenses positioned to improve the uniformity of UV radiation emitted from the LEDs towards the phototherapy zone, and wherein each individual TIR lens generally corresponds to an individual LED in the array of LEDs.

6. The home phototherapy system of claim 4 wherein the optical component includes an array of optical lenses positioned to collimate UV radiation emitted from the LEDs, and wherein each individual optical lens in the array of optical lenses generally corresponds to an individual LED in the array of LEDs.

7. The home phototherapy system of claim 4 wherein the optical component comprises an array of reflectors positioned to improve the uniformity of UV radiation emitted from the LEDs in the phototherapy zone, and wherein each individual reflector generally corresponds to an individual LED in the array of LEDs.

8. The home phototherapy system of claim 1 wherein the array of UV light emitters comprises a microplasma film having an array of microcavities configured to emit UV radiation.

9. The home phototherapy system of claim 1 wherein defining the dosing protocol includes:
determining, based on inputs from the user, a skin type associated with the user; and
determining, based on the skin type associated with the user, an initial dosage of the phototherapeutic UV radiation is configured to limit UV exposure to 0.5-0.7 MED based on the skin type associated with the user.

10. The home phototherapy system of claim 1 wherein defining the dosing protocol includes:
receiving inputs from the user to obtain information related to the user's reaction to a first dosage of the phototherapeutic UV radiation;
determining whether the user experienced erythema;
wherein:
if the user experienced erythema, determining an updated dose protocol to deliver a second dosage of the phototherapeutic UV radiation smaller than the first dosage, and
if the user did not experience erythema, determining the updated dose protocol to deliver a third dosage of the phototherapeutic UV radiation equal to or greater than the first dosage.

11. The home phototherapy system of claim 1 wherein the dose controller is further configured to execute an authentication protocol, the authentication protocol including—
receiving input credentials from the user;
authenticating the user using a registered user system;
determining, based at least partially on the result of the user authentication, whether to communicate with the UV-emitting device to power the UV light assembly on;
determining, based at least partially on a last access from the user, whether to communicate with the UV-emitting device to power the UV light assembly on; and
indicating to the user whether the UV light assembly will be powered on.

12. The home phototherapy system of claim 1 wherein the optical component is configured to direct the phototherapeutic UV radiation outwardly toward the phototherapy zone such that the phototherapeutic UV radiation has a uniform irradiance in the phototherapy zone.

13. The home phototherapy system of claim 1 wherein the phototherapy zone is between a first distance from the housing and a second distance from the housing larger than the first distance, and wherein the optical component is configured to direct the phototherapeutic UV radiation outwardly such that the phototherapeutic UV radiation has an irradiance that diverges by less than 10% between the first distance and the second distance.

14. A phototherapy system for producing vitamin D via skin exposure to ultraviolet (UV) radiation, the phototherapy system comprising:
an UV-emitting device having:
a housing with an active surface;
a UV light assembly carried by the housing and positioned to emit positioned to emit phototherapeutic UV radiation having a peak wavelength between 293 nm and 299 nm away from the active surface;
an optical component disposed over the UV light assembly and configured to collimate the phototherapeutic UV radiation to improve an average distribution of the phototherapeutic UV radiation exiting the UV-emitting device; and
an electronics controller in operably coupled to the UV light assembly;
an application executable on an electronic device, the application configured to use the electronic device to communicate with the electronics controller to provide a dosing protocol to the electronics controller, wherein the dosing protocol defines a dosage of the phototherapeutic UV radiation for a user; and
a dose controller communicatively coupled to the application, wherein the dose controller is configured to execute a dose-defining protocol to define the dosing protocol and communicate the dosing protocol to the application.

15. The phototherapy system of claim 14 wherein the application is further configured to execute an authentication protocol, the authentication protocol including—
receiving input credentials from the user through the electronic device;
authenticating the user using a registered user system;
determining, based at least partially on the result of the user authentication, whether to communicate with the UV-emitting device to power the UV light assembly on; and
indicating to the user whether the UV light assembly will be powered on.

16. The phototherapy system of claim 15 wherein the authentication protocol further including determining, based at least partially on a last access by the user, whether to communicate with the UV-emitting device to power the UV light assembly on.

17. The phototherapy system of claim 14 wherein the dose-defining protocol includes:
receiving inputs from the user to obtain information related to the user's reaction to a first dosage of the phototherapeutic UV radiation;
determining whether the user experienced erythema;
wherein:
if the user experienced erythema, determining an updated dose protocol to deliver a second dosage of the phototherapeutic UV radiation smaller than the first dosage, and
if the user did not experience erythema, determining the updated dose protocol to deliver a third dosage of the phototherapeutic UV radiation equal to or greater than the first dosage.

18. The phototherapy system of claim 14 wherein the UV-emitting device further comprises a proximity sensor positioned to detect a distance of the user away from the active surface while the UV light assembly is powered one, and wherein the electronics controller is operably coupled to the proximity sensor to power the UV light assembly off if the distance of the user is below a predetermined threshold for a predetermined period.

19. A method for operating a phototherapy system for producing vitamin D via skin exposure to ultraviolet (UV) radiation, the method comprising:
executing a dose-determining protocol, wherein the dose-determining protocol includes—
receiving, from a user of the home phototherapy system, inputs related to a skin type for the user;
determining, from the inputs related to the user's skin type, a minimal erythemal dose (MED) associated the user; and
determining, based on the MED associated the user, a dosing protocol to deliver an initial dosage of phototherapeutic UV radiation for the user; and
sending the dosing protocol to a UV-emitting device in the phototherapy system.

20. The method of claim 19 wherein the dose-determining protocol further includes—
receiving, from the user, inputs related to the user's reaction to the initial dosage of the phototherapeutic UV radiation;
determining whether the user experienced erythema;
if the user experienced erythema, determining an updated dose protocol to deliver a second dosage of the phototherapeutic UV radiation smaller than the initial dosage, and
if the user did not experience erythema, determining the updated dose protocol to deliver a third dosage of the phototherapeutic UV radiation greater than the initial dosage.

21. The method of claim 19, further comprising executing a user authentication protocol, wherein the user authentication protocol including—
receiving, from the user, credentials specific to the user;
authenticating the user using a registered user system;
determining, based at least partially on the result of the user authentication, whether to send the dosing protocol to the UV-emitting device; and
providing, to the user, an indication of whether the dosing protocol will be sent to the UV-emitting device.
